(19) United States
(12) Patent Application Publication
(10) Pub. No.: US 2026/0315685 A1
(43) Pub. Date: Oct. 15, 2026

(54) DEVICES AND METHODS FOR DELIVERING RECONSTITUTED MEDICAMENTS

(71) Applicant: kaleo, Inc., Glen Allen, VA (US)

(72) Inventors: Paul F. MEYERS, Fishers, IN (US); Michael J. ROE, Powhatan, VA (US)

(73) Assignee: kaleo, Inc., Glen Allen, VA (US)

(21) Appl. No.: 19/485,626

(22) PCT Filed: May 20, 2024
(86) PCT No.: PCT/US2024/030246

§ 371 Date: Nov. 18, 2025

Related U.S. Application Data
(60) Provisional application No. 63/468,153, filed on May 22, 2023.

(51) Int. Cl. A61M 5/14; A61M 5/142; A61M 5/145; A61M 5/24

(52) U.S. Cl. CPC A61M 5/1409; A61M 5/14244; A61M 5/14526; A61M 5/2466; A61M 2005/14272; A61M 2005/247

(57) ABSTRACT

Devices and methods for delivering reconstituted medicaments are described herein. A medicament container assembly is disposed within the medicament cavity of a delivery device. The medicament container assembly has a first volume and a second volume, which are fluidically isolated. The first volume contains a first substance, and the second volume contains a second substance. A mixing actuator is movable between a first mixing actuator position and a second mixing actuator position to cause the production of a first pressurized gas, which places the first volume in fluid communication with the second volume. An energy storage member is configured to produce a second pressurized gas to convey a reconstituted medicament from the medicament container assembly.

### CROSS-REFERENCE TO RELATED APPLICATIONS

This application claims benefit of priority to U.S. Provisional Application No. 63/468,153, entitled “Devices and Methods for Delivering Reconstituted Medicaments,” filed May 22, 2023, which is incorporated herein by reference in its entirety.

### BACKGROUND

The embodiments described herein relate generally to medicament containers and medicament delivery devices, and more particularly to a medicament delivery device for reconstituting a medicament and delivering the reconstituted medicament into a patient.

Exposure to certain substances, such as, for example, peanuts, shellfish, bee venom, certain drugs, toxins, and the like, can cause allergic reactions in some individuals. Such allergic reactions can, at times, lead to anaphylactic shock, which can cause a sharp drop in blood pressure, hives, and/or severe airway constriction. Similarly, certain medical conditions, such as diabetes, can result in rapid changes or reductions in blood glucose levels, resulting in an emergency condition requiring immediate treatment. As another example, exposure to toxins or nerve agents (e.g., sarin or other organophosphorus compounds) requires immediate treatment. Accordingly, responding rapidly to mitigate the effects from such conditions or exposures is important to prevent injury and/or death. For example, in certain situations, an injection of epinephrine (i.e., adrenaline) can provide substantial and/or complete relief from the allergic reaction. In other situations, for example, an injection of an antidote to a toxin, such as atropine, pralidoxime and midazolam, can greatly reduce and/or eliminate the harm potentially caused by the exposure. Similarly, an injection of glucagon can reduce and/or eliminate the harm potentially caused by reduced blood glucose levels in individuals experiencing a hypoglycemic emergency.

Because emergency medical facilities are not always available when an individual is suffering from such emergencies or medical conditions, some individuals carry an auto-injector, a rescue inhaler, or the like to rapidly self-administer a medicament in response to such emergency medical conditions. Some known auto-injectors include a vial containing a liquid medicament and a spring loaded needle to automatically penetrate the user's skin and inject the medicament. The storage of certain medicaments in a liquid form, however, can result in a shorter shelf life and/or an unstable medicament. Accordingly, some known auto-injectors include a vial containing a first medicament that is separated from a second medicament. Such auto-injectors are often referred to as “wet/dry” auto-injectors, because one substance is often a liquid (e.g., water or another solvent) and the other substance can be substantially solid or dry (e.g., lyophilized glucagon powder). Lyophilization is also known as “freeze drying.” In use, the first medicament and the second medicament must be mixed prior to injection.

Some known wet/dry injectors, however, require multiple steps to reconstitute the dry medicament and are therefore not suited for rapid deployment. For example, some known wet/dry injector require that the user manually actuate a mixing mechanism prior to injection (e.g., by twisting a portion of the device to complete the mixing step). Such configurations can, however, result in incomplete mixing and/or an injection occurring without mixing. As another example, the operation of some known wet/dry delivery systems includes manually inserting the needle into the skin prior to activation and subsequent medicament delivery. The operation of such configurations may also include separately attaching a needle to prepare the device for injection, resulting in a delay in delivery of the medicament. Moreover, such configurations can be complicated, making them difficult for a user to operate during an emergency or by an individual without medical training.

Some known wet/dry injectors require that a user manually perform a number of operations to prepare the injector for use. For example, some known wet/dry injectors require that the user vent and/or purge a portion of air included in the medicament container (e.g., mixed with or a part of the glucagon powder). In some instances, such known injectors are generally oriented in a predetermined manner (e.g., with the needle end facing upward) during the mixing process and/or prior to injection to facilitate the venting process (also referred to as “priming”). The use of such injectors, however, may not be practical in certain emergency situations, such as, for example, in a combat setting.

Some known wet/dry injectors employ a single mechanism to automatically mix and inject the medicaments contained therein. Although simpler to use, such known devices may not properly deliver the correct dose. For example, because the mixing operation and the injection operation are actuated together in such configurations, the medicament can sometimes be injected prior to the completion of the mixing operation and/or prior to the injector being properly positioned for the injection operation.

Additionally, although many known auto-injectors are carried by individuals in ordinary walks of life, such known devices may not be suitable for storage, transportation, or use in extreme periods for long durations of time. For example, some known auto-injectors may not be able to withstand high altitudes, temperature extremes, or shock (e.g., from dropping the device) that accompany use in military applications.

Thus, a need exists for improved medicament delivery devices to improve the procedures for mixing a medicament and accurately delivering the desired dose of reconstituted medicament.

### SUMMARY

Medicament delivery devices for reconstituting a medicament and delivering the medicament are described herein. In some embodiments, an apparatus (e.g., the medicament delivery device) includes a housing having an inner wall that defines a first gas chamber, a second gas chamber, and a medicament cavity. The medicament cavity is in fluid communication with the first gas chamber and the second gas chamber. A medicament container assembly is disposed within the medicament cavity. The medicament container assembly contains a medicament and has a first volume and a second volume. The first volume is fluidically isolated from the second volume. The first volume contains a first substance, and the second volume contains a second substance. A mixing actuator is positioned within the first gas chamber. The mixing actuator is movable within the first gas chamber between a first mixing actuator position and a second mixing actuator position. The movement of the mixing actuator from the first mixing actuator position towards the second mixing actuator position causes production of a first pressurized gas to place the first volume in fluid communication with the second volume. The apparatus also includes an energy storage member disposed in communication with the second gas chamber. The energy storage member is configured to produce a second pressurized gas to convey the medicament from the medicament container assembly on a condition that the energy storage member is actuated to release a potential energy stored therein.

In some embodiments, the first gas chamber has a first pneumatic volume on a condition that the mixing actuator is at the first mixing actuator position. The first gas chamber has a second pneumatic volume on a condition that the mixing actuator is at the second mixing actuator position, and the second pneumatic volume is less than the first pneumatic volume.

In some embodiments, air within the first gas chamber has a first pressure on a condition that the mixing actuator is at the first mixing actuator position. The air within the first gas chamber has a second pressure on a condition that the mixing actuator is at the second mixing actuator position. The second pressure is greater than the first pressure.

In some embodiments, the apparatus includes a spring coupled between the housing and a distal end portion of the mixing actuator. The spring is configured to urge the mixing actuator towards the second mixing actuator position.

In some embodiments, the mixing actuator includes a lock tab that is positioned to engage the housing to retain the mixing actuator at the first mixing actuator position.

In some embodiments, the apparatus includes a safety member removably coupled to the housing. The safety member includes a release member configured to actuate the spring on a condition that the safety member is decoupled from the housing.

In some embodiments, an actuator seal surrounds a proximal end portion of the mixing actuator. The actuator seal is positioned between the proximal end portion of the mixing actuator and a wall of the first gas chamber.

In some embodiments, a one-way valve is positioned fluidically between the first gas chamber and the medicament cavity. The one-way valve is configured to preclude a flow of gas from the medicament cavity into the first gas chamber.

In some embodiments, the medicament container assembly includes a container body, a first elastomeric member, and a second elastomeric member. The first elastomeric member is disposed within a proximal end portion of the container body. The second elastomeric member is disposed within the container body distal to the first elastomeric member. The first elastomeric member and the second elastomeric member define at least in part the first volume containing the first substance. The second elastomeric member and a distal portion end portion of the container body define the second volume containing the second substance.

In some embodiments, the container body defines a bypass channel. The first volume is fluidically isolated from the second volume on a condition that the second elastomeric member is at a first longitudinal position. The first volume is fluidically coupled to the second volume on a condition that the second elastomeric member is at a second longitudinal position. The second longitudinal position is distal to the first longitudinal position, and the second longitudinal position is longitudinally aligned with the bypass channel.

In some embodiments, an apparatus (e.g., the medicament delivery device) includes a housing that has an inner wall that defines a medicament cavity and a mixing protrusion. A medicament container assembly is disposed within the medicament cavity. The medicament container assembly includes a container body containing a medicament. The medicament container assembly has a first volume and a second volume. The first volume is fluidically isolated from the second volume. The first volume contains a first substance, and the second volume contains a second substance. A needle is aligned with the medicament container assembly. The needle has a separation from a distal end portion of the container body. The apparatus also includes a carrier at least partially surrounding the container body and the needle. The carrier is movable within the housing between a first carrier position and a second carrier position. The carrier maintains the separation on a condition that the apparatus is in a stored condition. Similarly, the carrier maintains a separation on a condition that the apparatus is in a mix condition. The medicament container assembly is in contact with the mixing protrusion and the first volume is in fluid communication with the second volume on a condition that the carrier is at the second carrier position.

In some embodiments, the mixing protrusion is axially aligned with the medicament container assembly.

In some embodiments, the mixing protrusion is positioned to exert a longitudinal force on the first elastomeric member in response to a transition of the carrier from the first carrier position to the second carrier position. The longitudinal force is configured to move the second elastomeric member from the first longitudinal position to the second longitudinal position.

In some embodiments, the apparatus includes an energy storage member disposed within the housing. The energy storage member is configured to produce a force to convey the medicament from the medicament container assembly on a transition of the apparatus from the mixed condition to an actuated condition.

In some embodiments, the container body is configured to develop a distal movement relative to the carrier in response to the force produced by the energy storage member. The distal movement of the container body places the second volume in fluid communication with the needle.

In some embodiments, the carrier includes a lock tab. The lock tab is positioned to engage the housing to retain the carrier at the first carrier position.

In some embodiments, a safety member removably coupled to the housing. The safety member includes a release member configured to actuate a spring operably coupled to the carrier on a condition that the safety member is decoupled from the housing. The actuation of the spring transitions the apparatus from the stored condition to the mix condition.

### BRIEF DESCRIPTION OF THE DRAWINGS

FIG. 1 is a front perspective view of a medicament delivery device according to an embodiment in a first (stored) configuration.

FIG. 2 is a side view of the medicament delivery device shown in FIG. 2 in the first (stored) configuration.

FIGS. 3A and 3B are perspective views of a medicament container assembly of the medicament delivery device shown in FIG. 1 when the medicament delivery device is in the first (stored) configuration (FIG. 3A) and when the medicament delivery device is being transitioned to a mix configuration (FIG. 3B).

FIG. 4 is a schematic illustration of a medicament delivery device according to an embodiment in a first (stored) configuration.

FIG. 5 is a schematic illustration of the medicament delivery device shown in FIG. 4 in a second (mixed) configuration.

FIG. 6 is a schematic illustration of the medicament delivery device shown in FIG. 4 in a third (actuated) configuration in which the medicament is reconstituted, a needle is in fluid communication with the reconstituted medicament, and the needle is extended prior to dispensing the medicament from the medicament delivery device.

FIG. 7 is a schematic illustration of the medicament delivery device shown in FIG. 4 in a fourth configuration following the dispensing of the reconstituted medicament from the medicament delivery device.

FIG. 8 is a cross-sectional view of a medicament delivery device according to an embodiment taken along line X-X in FIG. 2, the medicament delivery device being in the first (stored) configuration.

FIG. 9 is a cross-sectional view of the medicament delivery device shown in FIG. 8 in a second (mixed) configuration.

FIG. 10 is a cross-sectional view of the medicament delivery device shown in FIG. 8 in a third (actuated) configuration in which the medicament is reconstituted, a needle is in fluid communication with the reconstituted medicament, and the needle is extended prior to dispensing the medicament from the medicament delivery device.

FIG. 11 is a cross-sectional view of the medicament delivery device shown in FIG. 8 in a fourth (dispensed) configuration.

FIG. 12 is a cross-sectional view of the medicament delivery device shown in FIG. 8 in a fifth (retracted) configuration.

FIG. 13 is a schematic illustration of a medicament delivery device according to an embodiment in a first (stored) configuration.

FIG. 14 is a schematic illustration of the medicament delivery device shown in FIG. 13 in a second (mixed) configuration.

FIG. 15 is a schematic illustration of the medicament delivery device shown in FIG. 13 in a third (actuated) configuration in which the medicament is reconstituted, a needle is in fluid communication with the reconstituted medicament, and the needle is extended prior to dispensing the medicament from the medicament delivery device.

FIG. 16 is a schematic illustration of the medicament delivery device shown in FIG. 13 in a fourth configuration following the dispensing of the reconstituted medicament from the medicament delivery device.

FIG. 17 is a cross-sectional view of a medicament delivery device according to an embodiment taken along line X-X in FIG. 2, the medicament delivery device being in the first (stored) configuration.

FIG. 18 is a cross-sectional view of the medicament delivery device shown in FIG. 17 in a second (mixed) configuration.

FIG. 19 is a close-up cross-sectional view of a distal portion of the medicament container assembly shown in FIG. 17 in the first (stored) configuration and FIG. 18 in the second (mixed) configuration depicting the separation between a needle and the distal portion of the container body.

FIG. 20 is a cross-sectional view of the medicament delivery device shown in FIG. 17 in a third (actuated) configuration in which the medicament is reconstituted, the needle is in fluid communication with the reconstituted medicament, and the needle is extended prior to dispensing the medicament from the medicament delivery device.

FIG. 21 is a cross-sectional view of the medicament delivery device shown in FIG. 17 in a fourth (dispensed) configuration.

FIG. 22 is a cross-sectional view of the medicament delivery device shown in FIG. 17 in a fifth (retracted) configuration.

### DETAILED DESCRIPTION

Medicament container assemblies that contain a dry medicament and a solvent are described herein. The medicament container assemblies can be included within any of the medicament delivery devices described herein. The described medicament container assemblies include features (e.g., shaped bypass channels) and are configured to be manipulated (e.g., via the desired force exerted on the plungers) such that the dry medicament is reconstituted rapidly (quasi-instantaneously) by the flow of the solvent into the dry medicament without requiring additional steps (e.g., shaking, inverting the device, or the like). The medicament delivery devices described herein include a pair of energy sources. The first energy source (e.g., spring) is actuated automatically by the removal of a safety member from the housing of the medicament delivery device. The actuation of the first energy storage device establishes the flow of the solvent into the dry medicament (e.g., mixes the medicament delivery device by reconstituting medicament contained in the medicament container assembly). The second energy source can then be actuated (e.g., by pressing a base of the medicament delivery device against an injection location) to dispense the reconstituted medicament from the medicament container assembly.

Accordingly, the medicament delivery devices described herein are suitable for use in a variety of emergency situations where more complex, multi-step procedures are not desirable. Such situations can include, for example, drug delivery in a decentralized setting, in a battlefield setting, in a mass delivery setting (vaccinations, delivery of antidote for nerve agents, or the like).

Additionally, medicament delivery devices that are well suited to be stored for long durations (up to 2 years, up to 5 years, up to 10 years) in rugged environments are described herein. The medicament delivery devices described herein are configured with multiple different levels of safety to prevent premature actuation or inadvertent piercing of the medicament container (e.g., due to being dropped, shaken, stored in environments with changing ambient conditions, etc.).

As used in this specification, the singular forms “a,” “an” and “the” include plural referents unless the context clearly dictates otherwise. Thus, for example, the term “a member” is intended to mean a single member or a combination of members, “a material” is intended to mean one or more materials, or a combination thereof.

As used herein, the term “medicament” includes any constituent of a therapeutic substance. A medicament can include such constituents regardless of their state of matter (e.g., solid, liquid or gas). Moreover, a medicament can include the multiple constituents that can be included in a therapeutic substance in a mixed state, in an unmixed state and/or in a partially mixed state. A medicament can include both the active constituents and inert constituents of a therapeutic substance. Accordingly, as used herein, a medicament can include non-active constituents such as, water, colorant or the like.

The term “about” when used in connection with a referenced numeric indication means the referenced numeric indication plus or minus up to 10 percent of that referenced numeric indication. For example, “about 100” means from 90 to 110.

As used herein, the words “proximal” and “distal” refer to direction closer to and away from, respectively, an operator of the medical device. Thus, for example, the end of the medicament delivery device contacting the patient's body would be the distal end of the medicament delivery device, while the end opposite the distal end would be the proximal end of the medicament delivery device. As another example, the distal end portion of a medical injector is the end from which a needle or delivery member extends during the delivery event.

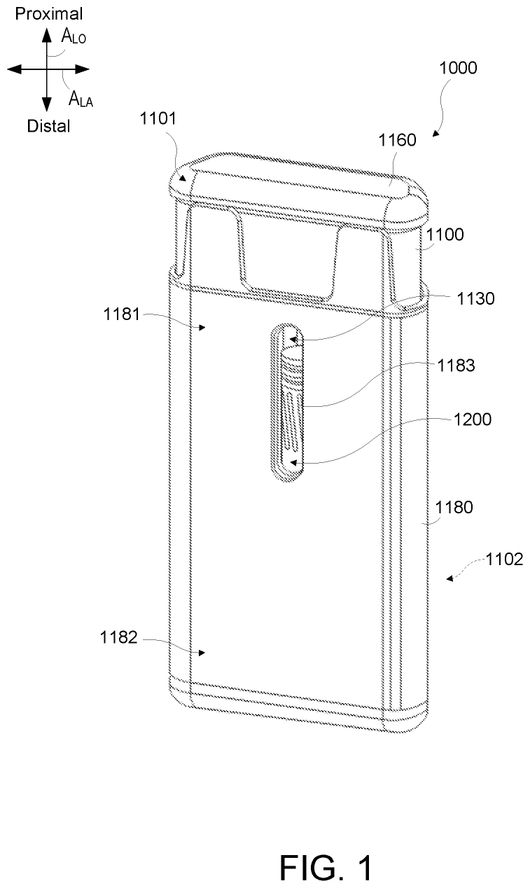

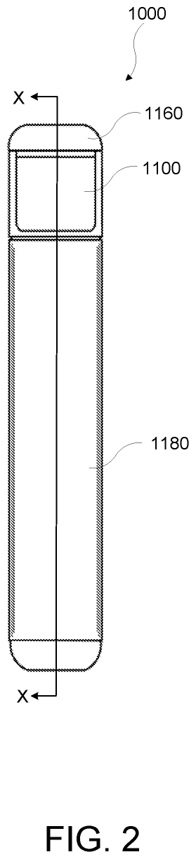

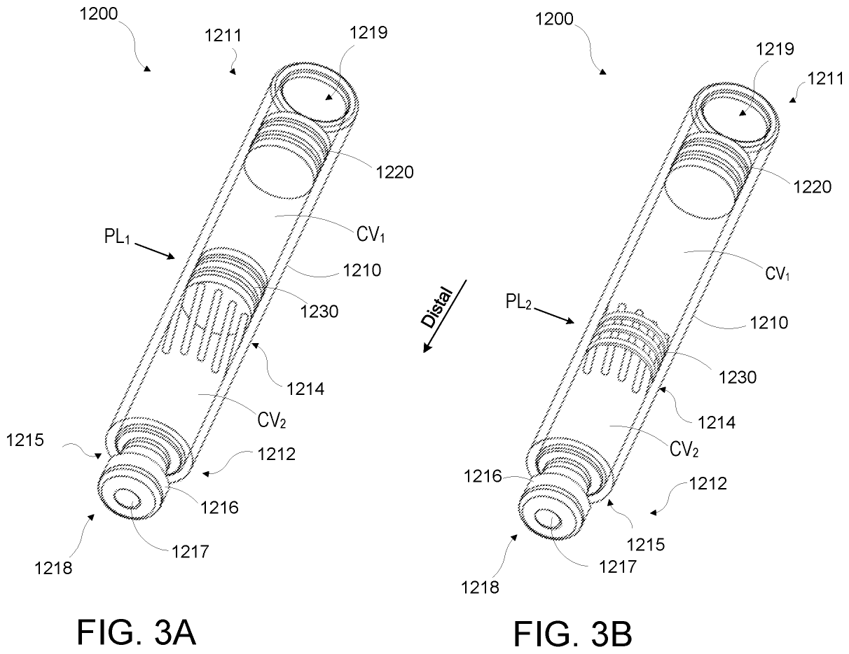

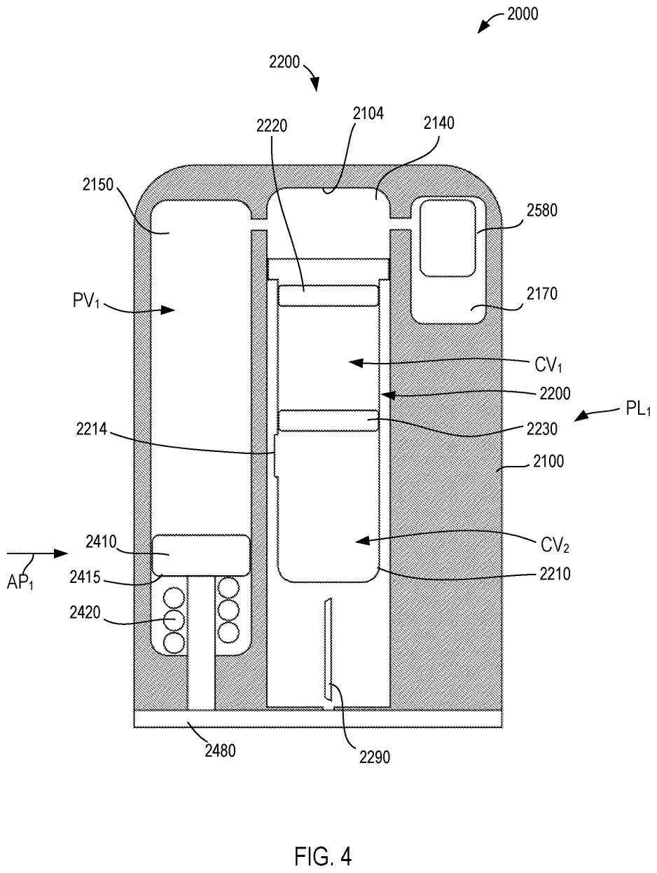

FIGS. 1 and 2 depict a medical injector 1000 (also referred to herein as “medicament delivery device,” “injector,” “apparatus,” or “drug product”) in a stored configuration (i.e., prior to use) according to an embodiment. The medical injector 1000 includes a cover 1180, a housing 1100, a safety member (not shown), a system actuation assembly (not shown), a medicament container assembly 1200 (FIGS. 3A and 3B), and a pair of energy sources (e.g., similar to the spring 2420 and the energy storage member 2580 depicted in FIGS. 4-7 or any other energy source described herein). The housing 1100 defines a longitudinal axis A_{LO} and a lateral axis A_{LA} orthogonal thereto for the medical injector 1000. As shown, the housing 1100 has a proximal end portion 1101 and a distal end portion 1102. The housing 1100 defines a pair of status indicator apertures 1130 disposed on a front side and a rear side of the housing 1100 (e.g., opposite sides of the housing 1100), which are configured to allow a patient to monitor the status and/or contents of the medicament container assembly 1200 contained within the housing 1100. For example, by visually inspecting the status indicator apertures 1130, a patient can determine whether the medicament container assembly 1200 contains a medicament and/or whether the medicament has been dispensed.

In some embodiments, the inner surface (e.g., an inner wall) of the housing 1100 can define various chambers and cavities. For example, the inner surface can define a medicament cavity (see e.g., medicament cavity 2140 in FIGS. 4-7) and at least one gas chamber (see e.g., the second gas chamber 2170 in FIGS. 4-7). The gas chamber is at least partially separated from the medicament cavity, but is in fluid communication with the medicament cavity to allow flow of pressurized gas via a gas passageway (not identified).

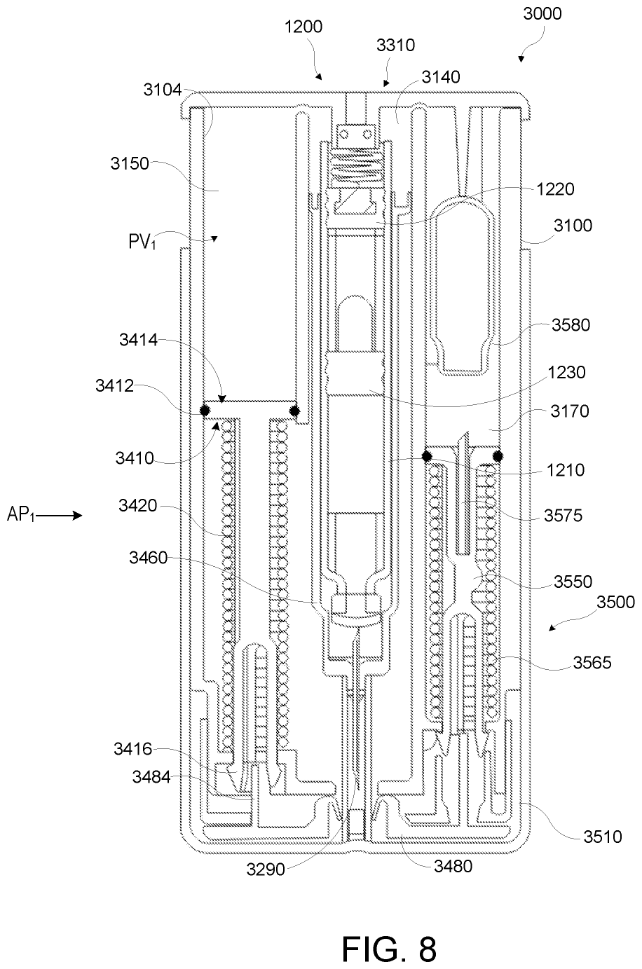

The medicament cavity is configured to receive the medicament container assembly 1200. The medicament container assembly 1200 is movable within the medicament cavity. In some embodiments, the medicament container assembly 1200 is movable both the proximal direction and in the distal direction. However, in some embodiments, the medicament container assembly 1200 is movable only in the distal direction. In some embodiments, the medicament container assembly 1200 is supported by a carrier (not shown). The carrier (see e.g., carrier 3460 in FIGS. 8-12) can include a seal member configured to form a substantially fluid tight seal with the inner surface of the housing 1100 defining the medicament cavity. Thus, a proximal portion of the medicament cavity forms a gas chamber that is substantially fluidically isolated by the seal member. The seal member can, for example, have two sealing portions: one that forms a seal against the container body 1210 (FIGS. 3A and 3B) and the other that forms a seal against the inner surface of the housing 1100. Upon a transition of the medical injector 1000 from a mixed configuration to an actuated configuration, a pressurized gas applies an actuation force in a distal direction against the seal member. The actuation force urges the two sealing portions outward (i.e., one sealing portion outward towards the container body 1210 and the other outward towards the housing 1100), which further enhances sealing. In other embodiments, however, the carrier can include multiple separate seals (e.g., O-rings) that form a fluid-tight seal with the housing 1100 and the container body 1210.

In some embodiments, the proximal portion of the medicament cavity can include a protrusion or stepped feature that limits movement of a retention portion of the carrier. On a condition that the medical injector 1000 is in the first configuration, the distal end portion 1212 (FIGS. 3A and 3B) of the container body 1210 can be secured within a retention portion of the carrier. Thus, the distal end portion of the medicament container assembly 1200 is within a coupling volume but spaced apart from the proximal tip of a needle. On a condition that the medical injector 1000 transitions from the mixed configuration to the actuated configuration, such as by pressing the base of the housing 1100 against an injection site, the carrier moves distally, and the gas pressure exerted causes the medicament container assembly 1200 to be pushed distally and released from the retention portion by an outward deformation of the retention portion. The optional proximal safety protrusion can contact the carrier in a manner to prevent inadvertent outward deformation of the retention portion when the device is in the first configuration. Similarly stated, in some embodiments, the inner wall of the housing 1100 that defines the medicament cavity can include a stepped portion that engages the carrier near the retention portion. This arrangement can limit the likelihood that the retention portion will inadvertently be deformed outward when the medical injector 1000 is in the storage configuration. This additional safety feature can be helpful to prevent the seal of the medicament container assembly 1200 from being punctured by the needle during conditions when the medical injector 1000 may be subjected to high external forces (e.g., vibratory forces, impact forces due to being dropped, etc.).

Although not depicted in FIGS. 1 and 2, the distal end portion 1102 of the housing 1100 can define various openings and apertures to facilitate the operation of the medical injector 1000. For example, the distal end portion 1102 can define a lock rod opening, a needle opening, and/or a system activation opening. The lock rod opening can receive a portion of a safety lock when the safety lock is coupled to the housing 1100. The needle opening is the opening through which the needle is disposed when the medical injector 1000 is actuated. The system activation opening receives a portion of the rod and allows the system actuator to be moved in a proximal direction relative to the housing 1100.

In some embodiments, the proximal end portion 1101 of the housing 1100 includes and/or is otherwise coupled to a proximal cap 1160. The proximal cap 1160 can include the retention members. The retention members of the proximal cap 1160 can be configured to receive and/or retain a gas container (e.g., energy storage member 3580 in FIGS. 8-12) that contains a pressurized gas. When the medical injector 1000 is actuated, pressurized gas from the gas container is conveyed from the gas cavity to the medicament cavity via the gas passageway. The proximal cap 1160 is coupled to the proximal surface of the housing 1100. In some embodiments, the proximal cap 1160 is fixedly coupled to the proximal surface via, for example, ultrasonic welding, adhesive, fasteners, and/or the like or a combination thereof. Moreover, a seal member can be disposed in a seal recess to form a substantially fluid tight seal between the proximal cap 1160 and the proximal surface of the housing 1100.

FIGS. 1 and 2 show the cover 1180 of the medical injector 1000. The cover 1180 can be any suitable configuration and can include any suitable feature to house, contain and/or protect portions of the medical injector 1000. The cover 1180 includes a proximal end portion 1181 and a distal end portion 1182. The cover 1180 also defines a cavity and a set of status windows 1183. The cavity of the cover 1180 is configured to receive at least a portion of the housing 1100. The status windows 1183 are disposed on opposite sides of the cover 1180 and are configured such that, when the portion of the housing 1100 is disposed within the cover 1180, the status windows 1183 of the cover 1180 are at least partially aligned with the corresponding status indicator aperture 1130 of the housing 1100. Thus, a user can visually inspect a portion of the medicament container assembly 1200 via the status windows 1183 of the cover 1180 and the status indicator apertures 1130 of the housing 1100. The cover 1180 also functions as a safety mechanism by preventing movement of the safety guard (e.g., safety member 3480 in FIGS. 8-12) and by preventing inward movement of the base (e.g., base 3510 in FIGS. 8-12).

FIGS. 3A and 3B are perspective views of the medicament container assembly 1200. The medicament container assembly 1200 includes a container body 1210 that has a proximal end portion 1211, a distal end portion 1212, and defining a proximal end opening 1219 and a distal end opening 1218.

The distal end portion 1212 of the container body 1210 includes a neck 1215 and a distal cap 1216 including a seal member 1217. The distal end portion 1212 can be configured to be at least partially disposed within a retention portion of a carrier, as described below. The distal cap 1216 can be, for example, a crimp seal or cap disposed about the distal end portion 1212 of the container body 1210. The seal member 1217 can be any suitable member, such as, for example, a septum, a valve, a frangible seal, and/or the like. In this manner, the seal member 1217 is configured to engage a surface of the container body 1210 and an inner surface of the distal cap 1216 to define a fluidic seal. The seal member 1217 can be configured to receive a puncturer (e.g., a needle similar to the needle 3290 in FIGS. 8-12) to dispense the medicament contained by the container body 1210.

The proximal end opening 1219 of the container body 1210 allows the inner volume 1213 to receive a first elastomeric member 1220 and a second elastomeric member 1230. The second elastomeric member 1230 is disposed within the container body 1210 distal to the first elastomeric member 1220. The first elastomeric member 1220 and the second elastomeric member 1230 are placed within the container body 1210 during a fill/finish process. The first elastomeric member 1220 and the second elastomeric member 1230 define, at least in part, a first volume CV₁ containing a first substance. The second elastomeric member 1230 and the distal end portion 1212 of the container body 1210 define a second volume CV₂ containing a second substance. For example, in some embodiments, the first elastomeric member 1220 and the second elastomeric member 1230 define a solvent volume, while the second elastomeric member 1230 and the distal end portion 1212 define a dry medicament volume. Said another way, the solvent volume is a volume disposed within the container body 1210 defined between the first elastomeric member 1220 and the second elastomeric member 1230. The dry medicament volume is a volume disposed within container body 1210 that can be defined between the second elastomeric member 1220 and the seal member 1217 disposed at the distal end portion 1212 of the container body 1210. Accordingly, the solvent volume and the dry medicament volume are defined by the positions of the first elastomeric member 1220 and the second elastomeric member 1230 relative to and/or within the container body 1210. In some embodiments, the solvent volume can contain a medicament solvent, such as, for example, an aqueous solvent. In some embodiments, the solvent can include an alcohol or a miscible organic solvent. In some embodiments, the solvent can include a surfactant. In some embodiments, the dry medicament volume can contain a dry medicament, which can be produced within the container body 1210 via a lyophilization process or alternatively, can be dry filled. The dry medicament can include any of the formulations and/or compositions described herein.

The first elastomeric member 1220 and the second elastomeric member 1230 can be of any design or formulation suitable for contact with the medicament (e.g., the solvent contained in the first volume CV₁ and/or a lyophilized or dry filled medicament contained in the second volume CV₂. For example, the elastomeric members 1220 and 1230 can be formulated to minimize any reduction in the efficacy of the medicament that may result from contact (either direct or indirect) between the elastomeric members 1220 and 1230 and the medicament. In some embodiments, the elastomeric members 1220 and 1230 can be made from and/or can include butyl rubber, such as chlorobutyl rubber, bromobutyl rubber, and/or the like. In some embodiments, the first elastomeric member 1220 and the second elastomeric member 1230 can be formulated to minimize any leaching or out-gassing of compositions that may have an undesired effect on the medicament. In other embodiments, the elastomeric members 1220 and 1230 can be formulated to maintain its chemical stability, flexibility and/or sealing properties when in contact (either direct or indirect) with the medicament over a long period of time (e.g., for up to six months, one year, two years, five years or longer).

The container body 1210 also defines one or more bypass channels 1214. The bypass channels 1214 are configured to facilitate the mixing and reconstitution of a dry medicament contained within the container body 1210, as described in further detail herein. In particular, the bypass channel 1214 is configured to place the first volume CV₁ and the second volume CV₂ in fluid communication with each other. In some embodiments, the bypass channels 1214 are positioned at a longitudinal location to minimize the size of the second volume CV₂ (e.g., the dry medicament volume), thus allowing for reduced amount of excess gas therein. Although shown as being a series of channels, in other embodiments, the bypass channel 1214 can be a singular channel bypass. Although the bypass channel 1214 is shown as being an internal bypass, in other embodiments, the bypass channel 1214 can be an external bypass (e.g., a portion that protrudes from the outer surface of the container body. Said another way, in some embodiments a bypass can be configured such that the outer diameter of the container body 1210 is substantially constant.

In some embodiments, the first volume CV₁ is fluidically isolated from the second volume CV₂ on a condition that the second elastomeric member 1230 is at a first longitudinal position PL₁ as depicted in FIG. 3A. However, the first volume CV₁ is fluidically coupled to the second volume CV₂ on a condition that the second elastomeric member 1230 is at a second longitudinal position PL₂ as depicted in FIG. 3B. The second longitudinal position PL₂ is distal to the first longitudinal position PL₁. The second longitudinal position PL₂ is also longitudinally aligned with the bypass channels 1214. In other words, on a condition that the second elastomeric member 1230 is at the same longitudinal position as at least a portion of the bypass channels 1214, the bypass channels 1214 place the first volume CV₁ in fluid communication with the second volume CV₂ thereby facilitating a reconstitution of the second substance (e.g., the dry medicament) by the first substance (e.g., the solvent).

As described in more detail below, in some embodiments, the second elastomeric member 1230 is configured to move in the distal direction from the first longitudinal position PL₁ in response to a force applied to the first elastomeric member 1220. The force applied to the first elastomeric member 1220 in the distal direction can be transmitted to the second elastomeric member 1230 via the first substance, which can be incompressible. When the distal movement of the second elastomeric member 1230 (in response to the force applied to the first elastomeric member 1220) places the second elastomeric member 1230 in longitudinal alignment with at least a portion of the bypass channels 1214 (i.e., at the second longitudinal position PL₂), the first substance can flow from the first volume CV₁, past the second elastomeric member 1230, and into second volume CV₂ via the bypass channels 1214. As such, the distal movement of the second elastomeric member 1230 can halt at the second longitudinal position PL₂ until the distal movement of the first elastomeric member 1220 brings the first elastomeric member 1222 contact with the second elastomeric member 1230.

In some embodiments, the medical injector 1000 can be an autoinjector that has a mixing actuator that is used to generate a first pressurized gas to reconstitute a medicament and has a piston-less delivery system in which the force exerted by a second pressurized gas can move the medicament container assembly relative to the housing and the elastomeric members relative to (e.g., within) the medicament container. For example, FIGS. 4-7 show a medical injector 2000 (also referred to as (medicament “delivery device,” “auto-injector,” “injector,” or “device”), according to an embodiment. The medical injector 2000 is a gas-powered auto-injector configured to utilize a first compressed gas to reconstitute the medicament contained within a medicament container assembly 2200 (e.g., a cartridge, a prefilled syringe, or other suitable container) and a second compressed gas to deliver a medicament, as described herein. The medical injector 2000 can include any of the features, structures, and/or elements described above with reference to the medical injector 1000. In some embodiments, the dry powder compositions and/or medicament container assemblies described herein can be used within any of the devices shown and described in U.S. Pat. No. 10,695,495, entitled “Devices and Methods for Delivering a Lyophilized Medicament,” filed on Sep. 20, 2017, U.S. Pat. No. 10,576,206, entitled “Auto-Injectors for Administration of a Medicament Within a Prefilled Syringe,” filed Dec. 19, 2017 (“the '206 Patent”) and International Patent Publication No. WO2020/140040, entitled “Devices and Methods for Delivery of Substances Within a Prefilled Syringe,” filed Dec. 27, 2019 (“the '0040 PCT”), each of which is incorporated herein by reference in its entirety.

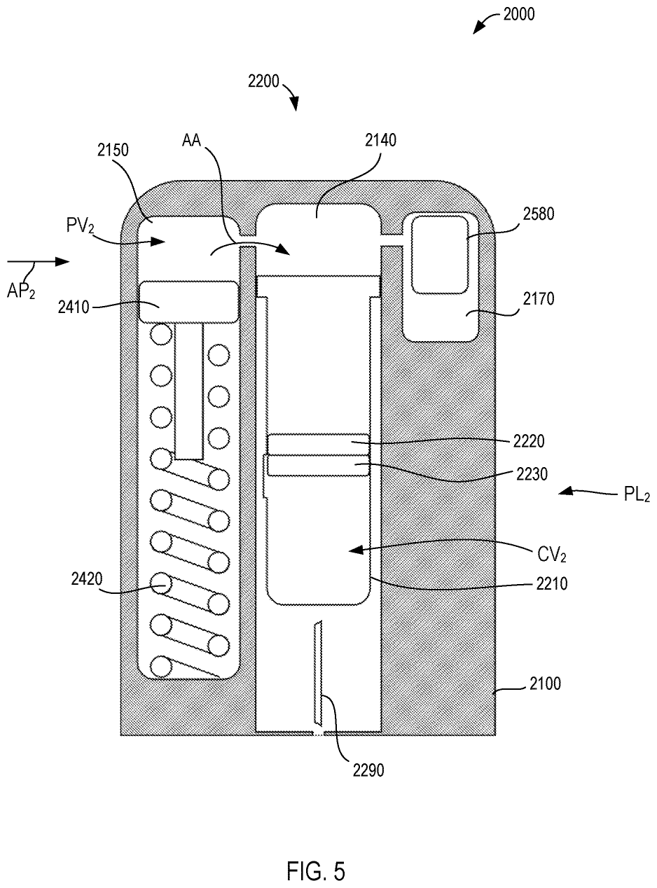

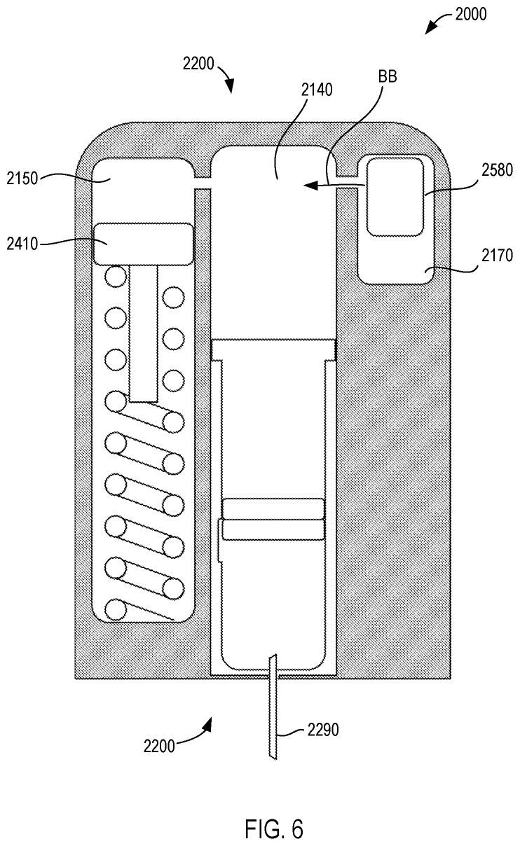

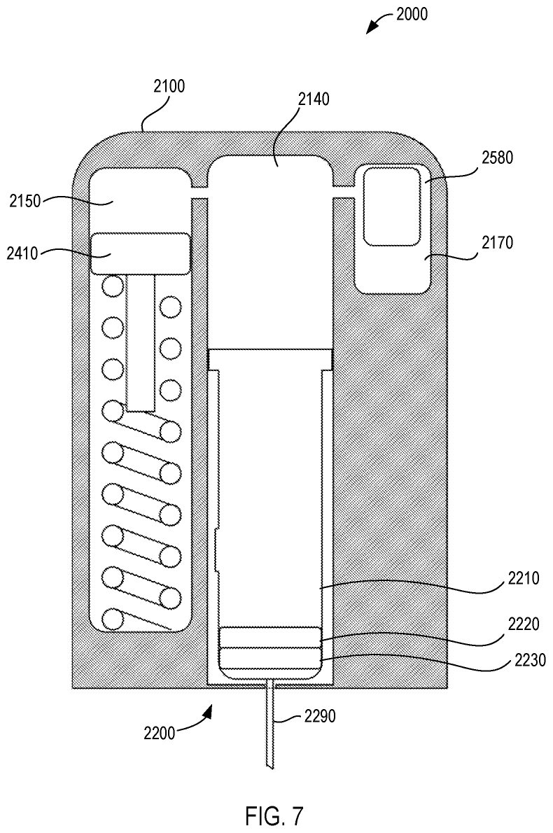

FIG. 4 depicts the medical injector 2000 in a first configuration. The first configuration can, for example, be a stored configuration. FIG. 5 depicts the medical injector 2000 following a transition from the first configuration to a second configuration. The second configuration can be a mixed configuration. The transition from the first configuration to the second configuration can be initiated by the removal of a safety member 2480 (FIG. 4). FIG. 6 depicts the medical injector 2000 following a transition from the second configuration (in which the medicament is partially or fully reconstituted) to a third configuration. The third configuration can be an actuated configuration in which a needle 2290 is placed in fluid communication with the reconstituted medicament within the container body 2210 and the needle 2290 is extended prior to dispensing the medicament from the medical injector 2000. FIG. 7 depicts the medical injector 2000 following a transition from the third configuration to a fourth configuration via the dispensing of the medicament from the medical injector 2000. In some embodiments, the fourth configuration can be followed by a transition to a fifth configuration in which the needle 2290 is retracted within the housing 2100.

As depicted, the medical injector 2000 includes a housing 2100 that has an inner wall 2104. The inner wall 2104 defines a first gas chamber 2150, a second gas chamber 2170, and a medicament cavity 2140. The medicament cavity 2140 is in fluid communication with the first gas chamber 2150 and the second gas chamber 2170. As such, the medicament cavity 2140 is configured to receive a first pressurized gas from the first gas chamber 2150 followed by a second pressurized gas from the second gas chamber 2170. The pressurized gas received by the medicament cavity 2140 can affect a longitudinal position of the medicament container assembly 2200 and/or the components thereof.

The medical injector 2000 includes a medicament container assembly 2200. The medicament container assembly 2200 can include any of the features, structures, and/or elements described herein with reference to the medicament container assembly 1200. The medicament container assembly 2200 is disposed within the medicament cavity 2140 and contains a dose of a medicament. As previously described in reference to medicament container assembly 1200, the medicament container assembly 2200 has a first volume CV₁ and a second volume CV₂. The first volume CVI contains a first substance (e.g., a solvent), while the second volume CV₂ contains a second substance (e.g., a dry component). In the first configuration, the first volume CV₁ is fluidically isolated from the second volume CV₂. The medicament container assembly includes a container body 2210, a first elastomeric member 2220 and a second elastomeric member 2230. The first elastomeric member 2220 is disposed within the proximal end portion of the container body 2210. The second elastomeric member 2230 is disposed within the container body 2210 distal to the first elastomeric member 2220. The first elastomeric member 2220 and the second elastomeric member 2230 define at least in part the first volume CV₁, while the second elastomeric member 2230 and a distal end portion of the container body 2210 defined the second volume CV₂. Additionally, the container body 2210 defines a bypass channel 2214. FIG. 4 depicts the second elastomeric member 2230 at a first longitudinal position PL₁ that results in the first volume CV₁ being fluidically isolated from the second volume CV₂. In contrast, FIG. 5 depicts the second elastomeric member 2230 following a distal movement to a second longitudinal position PL₂ that is longitudinally aligned with the bypass channel 2214. On a condition that the second elastomeric member 2230 is at the second longitudinal position PL₂, the first volume CV₁ is in fluid communication with the second volume CV₂ via the bypass channel 2214.

As depicted in FIGS. 4-7, the medical injector 2000 includes a mixing actuator 2410 positioned within the first gas chamber 2150. The mixing actuator 2410 is movable within the first gas chamber 2150. FIG. 4 depicts the mixing actuator 2410 at a first mixing actuator position AP₁. The mixing actuator 2410 is movable to a second mixing actuator position AP₂ as depicted in FIG. 5. In some embodiments, the movement of the mixing actuator 2410 can be in response to the urging of a spring 2420. The spring 2420 can be coupled between the housing 2100 and a distal end portion 2415 of the mixing actuator 2410. In other embodiments, the mixing actuator 2410 can urged towards the second actuator position AP₂ by any suitable biasing member or energy storage member (e.g., an elastic member, a pressurized member, or the like).

The movement of the mixing actuator 2410 from the first mixing actuator position AP₁ to the second mixing actuator position AP₂ produces a first pressurized gas. Said another way, the movement of the mixing actuator 2410 causes the compression of the air within the first gas chamber 2150 and the delivery of the compressed air to the medicament cavity 2140, as shown by the arrow AA. The first pressurized gas within the medicament cavity 2140 acts on the first elastomeric member 2220 resulting in a distal movement of the first elastomeric member 2220 and the second elastomeric member 2230 to place the first volume CV₁ in fluid communication with the second volume CV₂ (e.g., via the bypass channel 2214). As the solvent flows from the first volume CV₁ into the second volume CV₂, the first elastomeric member 2220 continues to move relative to the second elastomeric member 2230 until the first volume CV₁ is substantially gone (as shown in FIG. 5) and the solvent and the dry component are in the second volume CV₂. Said another way, the movement of the mixing actuator 2410 drives the mixing of the solvent and the dry component of the medicament within the container body 2210, thereby establishing the medical injector 2000 in a mixed condition from which the reconstituted medicament can be dispensed. Although the first elastomeric member 2220 and/or the second elastomeric member are configured to move distally in response to the first pressurized gas, the medicament container assembly 2200 remains at a fixed longitudinal position on a condition that the medical injector is in the first (stored) configuration, the second (mixed) configuration and during a transition therebetween.

As depicted in FIG. 4, the first gas chamber 2150 has a first pneumatic volume PV₁ on a condition that the mixing actuator 2410 is at the first mixing actuator position AP₁. The first pneumatic volume PV₁ can, for example, be the volume of the first gas chamber 2150 defined by the inner wall 2104 of the housing 2100 and the mixing actuator 2410 on a condition that the medical injector 2000 is in the stored configuration. Said another, in some embodiments the air within the first gas chamber 2150 has a first pressure on a condition that the mixing actuator 2410 is at the first mixing actuator position AP₁. As depicted in FIG. 5, the first gas chamber 2150 has a second pneumatic volume PV₂ on a condition that the mixing actuator 2410 is at the second mixing actuator position AP₂. The second pneumatic volume PV₂ can, for example, be the volume of the first gas chamber 2150 defined by the inner wall 2104 of the housing 2100 and the mixing actuator 2410 on a condition that the medical injector 2000 is in the mixed configuration. The air within the gas chamber 2150 has a second pressure on a condition that the mixing actuator 2410 is at the mixing actuator position AP₂. The second pressure is greater than the first pressure. In other words, the movement of the mixing actuator 2410 from the first actuator position AP₁ toward the second actuator position AP₂ reduces the volume of the first gas chamber 2150 and results in the production of the first pressurized gas. The resultant first pressurized gas flows from the first gas chamber 2150 to the medicament cavity 2140 (via the orifice, as shown by the arrow AA) to exert a force on the first elastomeric member 2220.

As depicted in FIG. 4, in some embodiments, the medical injector 2000 includes a safety member 2480. The safety member 2480 is removably coupled to the housing 2100. The safety member 2480 includes a release member (e.g., release member 3484 in FIG. 8). The release member is configured to actuate the spring 2420 on a condition that the safety member 2480 is decoupled from the housing 2100. Said another way, the removal of the safety member 2480 initiates a transition of the medical injector 2000 from the stored configuration to the mixed configuration by actuating the spring 2420 resulting in the reconstitution of the medicament within the medicament container assembly 2200. In other words, the medical injector 2000 is placed in a condition to be actuated to dispense the reconstituted medicament (e.g., a ready state) by the removal of the safety member 2480 and does not require additional inputs from an operator to prepare the medical injector 2000. In some embodiments, the safety member 2480 can also include features to limit the actuation of the energy storage member 2580 (described below) or otherwise prevent unintended actuation of the medical injector.

In some embodiments, the medical injector 2000 includes an energy storage member 2580. The energy storage member 2580 is disposed in communication with the second gas chamber 2170. The energy storage member 2580 is configured to produce a second pressurized gas on a condition that the energy storage member 2580 is actuated to release a potential energy stored therein. For example, in some embodiments, the energy storage member can be a compressed gas cylinder having any desired pressure (and thus, mass) of gas therein or a propellant container that produces a pressured gas upon release of the propellant therein. Upon actuation, the second pressurized gas flows from the second gas chamber 2170 to the medicament cavity 2140 (as shown by the arrow BB) to convey the medicament from the medicament container assembly 2200. The second pressurized gas has a greater pressure than the first pressurized gas.

In response to the second pressurized gas being conveyed into the medicament cavity 2140, the medicament container assembly 2200 is moved distally within the housing 2100 and/or relative to the needle 2290, thereby placing the needle in fluid communication with the second volume CV₂. Continued pressurization of the medicament cavity 2140 causes the needle 2290 and the medicament container assembly 2200 to move distally such that the needle 2290 extends from the housing (as shown in FIG. 6)—to cause insertion of the needle at the target location. After extension of the needle 2290, continued pressurization of the medicament cavity 2140 causes the first elastomeric member 2220 and the second elastomeric member 2230 to move distally within the container body to convey the reconstituted medicament out of the second volume CV₂ via the needle 2290 (as shown in FIG. 7).

In some embodiments, the medicament container assembly 1200 described above can be a component of an autoinjector that has a mixing actuator configured to generate a first pressurized gas to reconstitute a medicament and an energy storage member configured to deliver a second pressurized gas to dispense the medicament from the medicament container assembly. For example, FIGS. 8-12 show a medical injector 3000 (also referred to as (medicament “delivery device,” “auto-injector,” “injector,” or “device”), according to an embodiment. The medical injector 3000 is a gas-powered auto-injector configured to utilize a first compressed gas to reconstitute the medicament contained within the medicament container assembly 1200 and a second compressed gas to deliver a medicament, as described herein. The medical injector 3000 can include any of the features, structures, and/or elements described above with reference to any other medical injector described herein (e.g., the medical injector 1000 and the medical injector 2000).

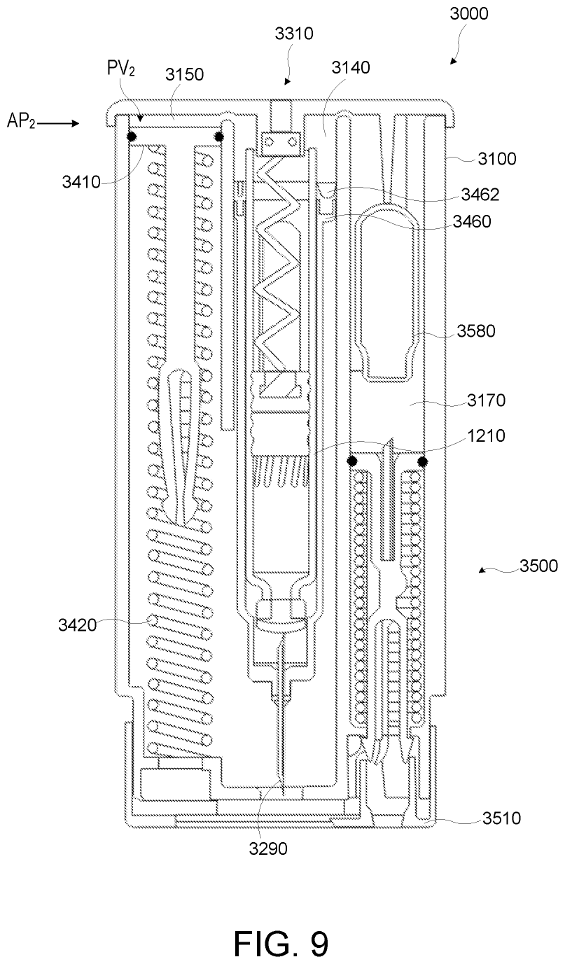

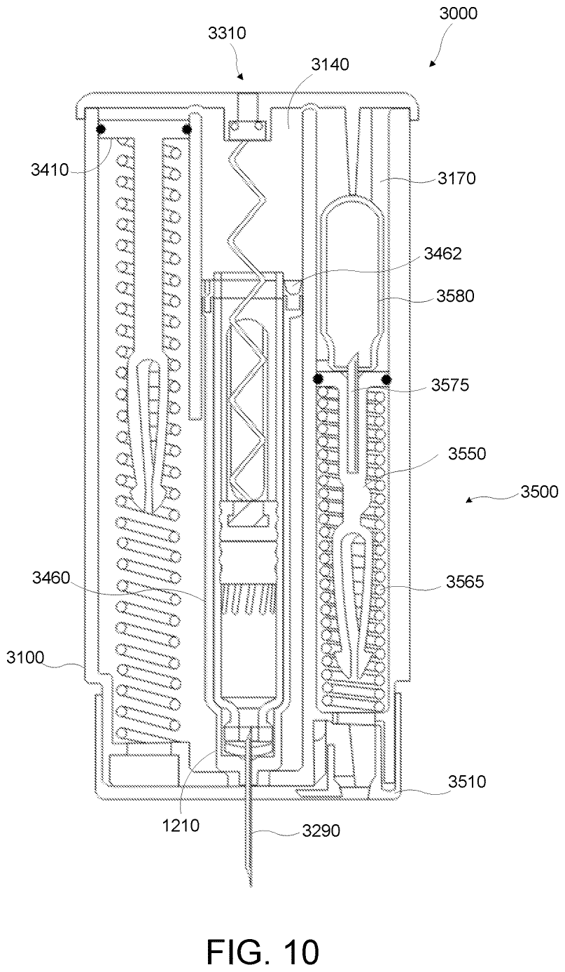

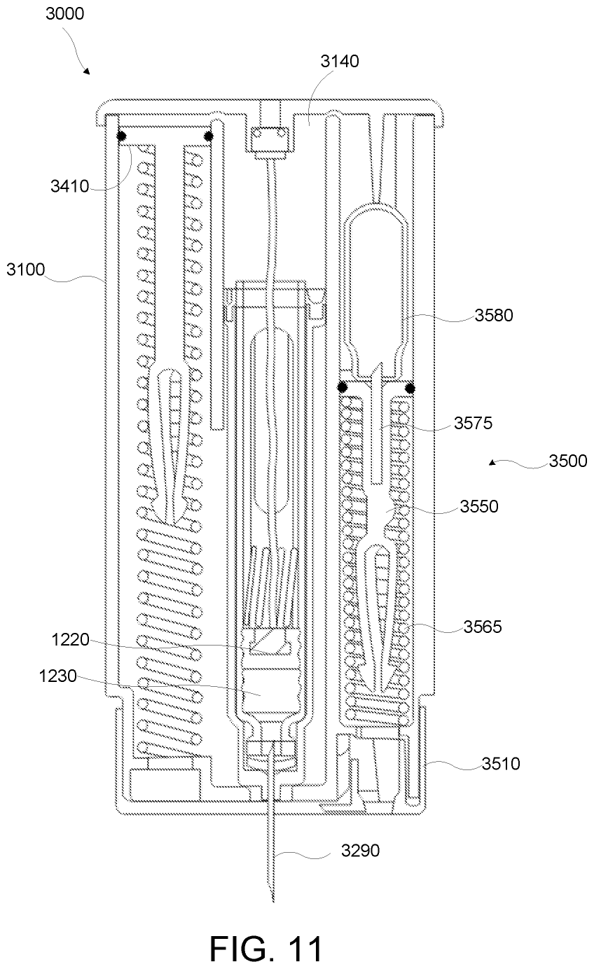

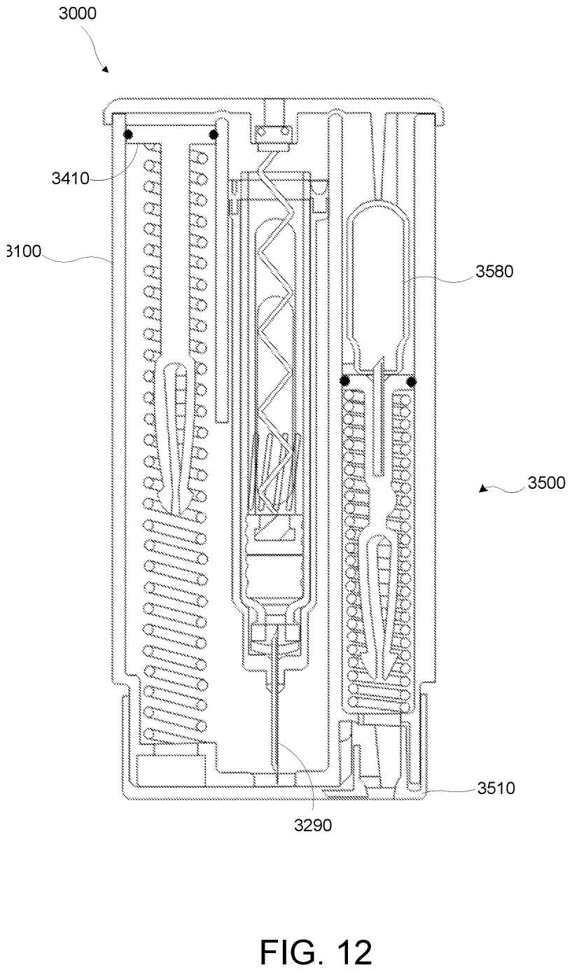

FIG. 8 depicts the medical injector 3000 in a first configuration. The first configuration can, for example, be a stored configuration. FIG. 9 depicts the medical injector 3000 following a transition from the first configuration to a second configuration. The second configuration can be a mixed configuration. The transition from the first configuration to the second configuration can be initiated by the removal of a safety member 3480. FIG. 10 depicts the medical injector 3000 following a transition from the second configuration to a third configuration. The third configuration can be an actuated configuration in which a needle 3290 is placed in fluid communication with the reconstituted medicament within the container body 1210 and the needle 3290 is extended prior to dispensing the medicament from the medical injector 3000. FIG. 11 depicts the medical injector 3000 following a transition from the third configuration to a fourth configuration via the dispensing of the medicament from the medical injector 3000. FIG. 12 depicts the medical injector 3000 in a fifth (retracted) configuration in which the needle 3290 is retracted within the housing 3100.

As depicted, the medical injector 3000 includes a housing 3100 that has an inner wall 3104. The inner wall 3104 defines a first gas chamber 3150, a second gas chamber 3170, and a medicament cavity 3140. The medicament cavity 3140 is in fluid communication with the first gas chamber 3150 and the second gas chamber 3170. As such, the medicament cavity 3140 is configured to receive a first pressurized gas from the first gas chamber 3150 followed by a second pressurized gas from the second gas chamber 3170. The pressurized gas received by the medicament cavity 3140 can affect a longitudinal position of the medicament container assembly 1200 and/or the components thereof.

The medical injector 3000 includes the medicament container assembly 1200 as described herein. The medicament container assembly 1200 is disposed within the medicament cavity 3140 and contains a dose of a medicament. The first elastomeric member 1220 is disposed within the proximal end portion of the container body 1210, and the second elastomeric member 1230 is disposed distal to the first elastomeric member 1220. As previously described the medicament container assembly 1200 has a first volume and a second volume. The first volume contains a first substance (e.g., a solvent), while the second volume contains a second substance (e.g., a dry component). As depicted in FIG. 8, the first volume is fluidically isolated from the second volume on a condition that the medical injector 3000 is in the stored configuration in which the second elastomeric member 1230 is at a first longitudinal position. In contrast, FIG. 9 depicts the second elastomeric member 1230 following a distal movement to a second longitudinal position that is longitudinally aligned with the bypass channel. On a condition that the second elastomeric member 1230 is at the second longitudinal position, the first volume is in fluid communication with the second volume via the bypass channel.

As depicted in FIGS. 8-12, the medical injector 3000 includes a mixing actuator 3410 positioned within the first gas chamber 3150. The mixing actuator 3410 is movable within the first gas chamber 3150. The mixing actuator 3410 can, for example, be configured as a piston, a plunger, or other similar structure. FIG. 8 depicts the mixing actuator 3410 at a first mixing actuator position AP₁. The mixing actuator 3410 is movable to a second mixing actuator position AP₂ as depicted in FIG. 9. In some embodiments, the movement of the mixing actuator 3410 can be in response to the urging of a spring 3420. The spring 3420 can be coupled between the housing 3100 and a distal end portion 3415 of the mixing actuator 3410.

In some embodiments, an actuator seal 3412 (e.g., an O-ring) surrounds a proximal end portion 3414 of the mixing actuator 3410. The actuator seal 3412 can be axially aligned with the mixing actuator 3410. As depicted in FIGS. 8-12, the actuator seal 3412 is positioned between the proximal end portion 3414 of the mixing actuator 3410 and a wall of the first gas chamber 3150 (e.g., a portion of the inner wall 3104 of the housing 3100 defining a gas cavity). As such, the actuator seal 3412 forms a fluid-tight seal with the housing 3100.

The movement of the mixing actuator 3410 from the first mixing actuator position AP₁ to the second mixing actuator position AP₂ produces a first pressurized gas. Said another way, the movement of the mixing actuator 3410 causes the compression of the air within the first gas chamber 3150 and the delivery of the compressed air to the medicament cavity 3140. The first pressurized gas within the medicament cavity 3140 acts on the first elastomeric member 1220 resulting in a distal movement of the first elastomeric member 1220 and the second elastomeric member 1230 to place the first volume in fluid communication with the second volume (e.g., via the bypass channel 1214; see FIGS. 3A and 3B). As the solvent flows from the first volume CV₁ into the second volume CV₂, the first elastomeric member 3220 continues to move relative to the second elastomeric member 3230 until the first volume CV₁ is substantially gone (as shown in FIG. 9) and the solvent and the dry component are in the second volume CV₂. Said another way, the movement of the mixing actuator 3410 drives the mixing of the solvent and the dry component of the medicament within the container body 1210, thereby establishing the medical injector 3000 in a mixed condition from which the reconstituted medicament can be dispensed. Although the first elastomeric member 1220 and/or the second elastomeric member 1230 are configured to move distally in response to the first pressurized gas, the medicament container assembly 1200 remains at a fixed longitudinal position on a condition that the medical injector is in the first (stored) configuration, the second (mixed) configuration and during a transition therebetween.

As depicted in FIG. 8, the first gas chamber 3150 has a first pneumatic volume PV₁ on a condition that the mixing actuator 3410 is at the first mixing actuator position AP₁. The first pneumatic volume PV₁ can, for example, be the volume of the first gas chamber 3150 defined by the inner wall 3104 of the housing 3100 and the mixing actuator 3410 on a condition that the medical injector 3000 is in the stored configuration. Said another, in some embodiments the air within the first gas chamber 3150 has a first pressure on a condition that the mixing actuator 3410 is at the first mixing actuator position AP₁. As depicted in FIG. 9, the first gas chamber 3150 has a second pneumatic volume PV₂ on a condition that the mixing actuator 3410 is at the second mixing actuator position AP₂. The second pneumatic volume PV₂ can, for example, be the volume of the first gas chamber 3150 defined by the inner wall 3104 of the housing 3100 and the mixing actuator 3410 on a condition that the medical injector 3000 is in the mixed configuration. The air within the gas chamber 3150 has a second pressure on a condition that the mixing actuator 3410 is at the mixing actuator position AP₂. The second pressure is greater than the first pressure. In other words, the movement of the mixing actuator 3410 from the first actuator position AP₁ toward the second actuator position AP₂ reduces the volume of the first gas chamber 3150 and results in the production of the first pressurized gas. The resultant first pressurized gas flows from the first gas chamber 3150 to the medicament cavity 3140 to exert a force on the first elastomeric member 1220.

In some embodiments, the mixing actuator 3410 includes a lock tab 3416. The lock tab 3416 can be a projection, a protrusion, and/or other structure having a surface configured to selectively engage the housing 3100. The lock tab 3416 is configured to retain the mixing actuator 3410 at the first mixing actuator position AP₁ on a condition that the medical injector 3000 is in the stored configuration. More specifically, the mixing actuator 3410 is disposed within the first gas chamber 3150 of the housing 3100 such that lock tab 3416 extends through an opening defined by the inner wall 3104 on a condition that the medical injector 3000 is in the stored configuration. Thus, the lock tab 3416 engages a portion of the inner wall 3104 and is maintained in contact therewith on the condition that the medical injector 3000 is in the stored configuration. In other words, lock tab 3416 engages a portion of the housing 3100 to limit proximal movement of the mixing actuator 3410 prior to actuation of the spring 3420.

As depicted in FIG. 8, in some embodiments, the medical injector 3000 includes a safety member 3480. The safety member 3480 is removably coupled to the housing 3100. The safety member 3480 includes a release member 3484 that is configured to actuate the spring 3420 on a condition that the safety member 3480 is decoupled from the housing 3100. As such, the engagement of the lock tab 3416 with the housing 3100 is maintained by the release member 3484 (which pushes the lock tabs 3416 away from each other and into contact with the housing) on a condition that the safety member 3480 is coupled to the housing 3100. Said another way, the removal of the safety member 3480 initiates a transition of the medical injector 3000 from the stored configuration to the mixed configuration by actuating the spring 3420 resulting in the reconstitution of the medicament within the medicament container assembly 1200. In other words, the medical injector 3000 is placed in a condition to be actuated to dispense the reconstituted medicament (e.g., a ready state) by the removal of the safety member 3480 and does not require additional inputs from an operator to prepare the medical injector 3000.

In some embodiments, the medical injector 3000 includes an energy storage member 3580. The energy storage member 3580 is disposed in communication with the second gas chamber 3170. The energy storage member 3580 is configured to produce a second pressurized gas on a condition that the energy storage member 3580 is actuated to release a potential energy stored therein. For example, in some embodiments, the energy storage member can be a compressed gas cylinder having any desired pressure (and thus, mass) of gas therein. Upon actuation, the second pressurized gas flows from the second gas chamber 3170 to the medicament cavity 3140 to convey the medicament from the medicament container assembly 1200. The second pressurized gas has a greater pressure than the first pressurized gas. As such, in some embodiments, a one-way valve is positioned fluidically between the first gas chamber 3150 and the medicament cavity 3140 to preclude a flow of the second pressurized gas from the medicament cavity 3140 into the first gas chamber 3150.

As depicted in FIGS. 8-12, the medicament container assembly 1200 is at least partially surrounded and supported by a carrier 3460. The carrier 3460 and the medicament container assembly 1200 are movable within the medicament cavity 3140 in the distal direction. Moreover, the carrier 3460 includes a seal member 3462 configured to form a substantially fluid tight seal with the inner wall 3104 of the housing 3100 defining the medicament cavity 3140. The seal member 3462 also seals the carrier 3460 and the container body 1210. Thus, the proximal portion of the medicament cavity 3140 forms a gas chamber that is substantially fluidically isolated by the seal member 3462. Specifically, the seal member 3462 has two sealing portions: one that forms a seal against the container body 1210 and the other that forms a seal against the inner surface of the housing 3100. In use, the second pressurized gas applies an actuation force in a distal direction against the seal member 3462. The actuation force urges the two sealing portions outward (i.e., one sealing portion outward towards the container body 1210 and the other outward towards the housing 3100), which further enhances sealing. In other embodiments, however, the carrier 3460 can include multiple separate seals (e.g., O-rings) that form a fluid-tight seal with the housing 3100 and the container body 1210. The seal member 3462 can maintain the medicament container assembly 1200 and the carrier 3460 in a fixed longitudinal position in the presence of the first pressurized gas. In other words, the friction force between the seal member 3462 and the inner wall 3104 of the housing 3100 can be greater than the magnitude of the first pressurized gas. As such, the first elastomeric member 1220 can move in the distal direction in response to the first pressurized gas while the medicament container assembly 1200 and the carrier 3460 remain in the fixed longitudinal position.

In some embodiments, the proximal portion of the medicament cavity 3140 can include a protrusion or stepped feature that limits movement of a retention portion of the carrier 3460. As depicted in FIGS. 8 and 9, when the medical injector 3000 is in the first configuration, the distal end portion of the container body 1210 is secured within a retention portion of the carrier 3460. Thus, the distal end portion of the medicament container assembly 1200 is within a coupling volume of the carrier 3460 but spaced apart from a proximal tip of the needle 3290. As shown in FIGS. 10 and 11, when the device is actuated the carrier 3460 moves distally, and the gas pressure exerted causes the medicament container assembly 1200 to be pushed distally and released from the retention portion (e.g., by an outward deformation of the retention portion). An optional proximal safety protrusion can contact the carrier in a manner to prevent an inadvertent release from the retention portion when the medical injector 3000 is in the first configuration and in the second configuration. Similarly stated, in some embodiments, the inner wall of the housing 3100 that defines the medicament cavity 3140 can include a stepped portion that engages the carrier 3460 near the retention portion. This arrangement limits the likelihood that retention portion will inadvertently be deformed outward when the medical injector 3000 is in the storage configuration and in the mixed configuration. This additional safety feature can be helpful to prevent the seal of the medicament container assembly 1200 from being punctured by the needle 3290 during conditions when the medical injector 3000 may be subjected to high external forces (e.g., vibratory forces, impact forces due to being dropped, etc.).

As depicted in FIGS. 8-12, the medical injector 3000 includes a base 3510. The base 3510 has a similar structure and function as the bases shown in International Patent Publication No. WO2017/004345, entitled “Auto-Injectors for Administration of a Medicament Within a Prefilled Syringe,” filed Jun. 30, 2016 (“the '4345 PCT”) and International Patent Publication No. WO2020/140040, entitled “Devices and Methods for Delivery of Substances Within a Prefilled Syringe,” filed Dec. 27, 2019 (“the '0040 PCT”), each of which is incorporated herein by reference in its entirety.

The medical injector 3000 can then be activated by moving the base 3510 from a first position, as depicted in FIG. 9, to a second position, as depicted in FIG. 10, to place the medical injector 3000 in the third (actuated) configuration. Similarly stated, the medical injector 3000 can be activated by the system actuator assembly 3500 (e.g., a puncturer release member 3550 and a spring 3565) by moving the base 3510 proximally relative to the housing 3100. The base 3510 is moved from its first position to its second position by placing the medical injector 3000 against a target surface (e.g., the body of the patient) and moving the base 3510 with respect to the housing 3100 in the proximal direction.

The proximal movement of the base 3510 from its first position to its second position actuates and/or otherwise releases the puncturer release member 3550. As such, the spring 3565 is allowed to transition from a first configuration (e.g., a compressed configuration) to a second configuration (e.g., a non-compressed configuration), thereby moving the puncturer release member 3550 within the second gas chamber 3170. Proximal movement of the puncturer release member 3550 causes a puncturer 3575 to puncture and/or pierce a portion of the energy storage member 3580 (e.g., a frangible seal or the like). After the gas container 3580 has been punctured, an actuating portion of a compressed gas flows from the energy storage member 3580 (e.g., gas container), into the second gas chamber 3170, and into the medicament cavity 3140. More specifically, with a seal of the puncturer release member 3550 forming a substantially fluid tight seal with the inner surface defining, at least in part, the second gas chamber 3170, the actuating portion of the compressed gas fills the second gas chamber 3170 and is forced through the gas passageway and into the medicament cavity 3140.

As the gas flows into the medicament cavity 3140, the gas applies gas pressure to the upper portion of the carrier 3460 (including the seal member 3462 (e.g., a carrier seal)) and the first elastomeric member 1220 within the container body 1210. This gas force (the insertion force) causes the container body 1210, the carrier 3460 and the needle 3290 to contemporaneously move within the housing 3100 in the distal direction. The movement of the needle 3290 in a distal direction causes the distal end portion of the needle 3290 to exit the housing 3100 and enter the body of a patient prior to administering the medicament.

Insofar as the medical injector 3000 is configured to dispense the reconstituted medicament contained within the medicament container assembly 1200, the medical injector 3000 is actuated from the second configuration, such as depicted in FIG. 9. Accordingly, as depicted in FIG. 10, the first elastomeric member 1220 can be in contact with the second elastomeric member 1230 such that the gas pressure of the second pressurized gas exerts a force sufficient to move both the elastomeric members 1220 and 1230 in the distal direction. The distal movement of the elastomeric members 1220 and 1230 generates a pressure upon the reconstituted medicament contained within the container body 1210, thereby allowing at least a portion of the medicament to flow out of the container body 1210 via the needle 3290, upon the transition from the third (actuated) configuration to the fourth (dispensed) configuration as shown in FIG. 11. Furthermore, when the elastomeric members 1220 and 1230 are disposed in a distal position within the container body 1210, the medical injector 3000 has delivered a dose of the reconstituted medicament.

As depicted in FIGS. 8-12, the medical injector 3000 includes a gas release assembly 3310. The gas release assembly 3310 expands as the first elastomeric member 1220 moves distally and serves to release a gas vent when the end of the injector stroke is reached. The gas release assembly 3310 a has similar structure and function as the gas release (or expandable) assemblies shown in the '4345 PCT and the '0040 PCT, each of which is incorporated herein by reference in its entirety.

FIG. 12 shows the medical injector 3000 in its retracted position (e.g., the fifth configuration). The retraction of the carrier 3460 and the needle 3290 occurs in response to the second pressurized gas within the medicament cavity 3140 of the housing 3100 being vented by the expansion of the gas release assembly 3310. Specifically, when the first elastomeric member 1220 reaches the desired position (i.e., its distal-most position corresponding to the end of injection), the gas release assembly 3310 actuates a valve to release the gas. Operation of the gas release assembly 3310 can be similar to that shown in the '4345 PCT and the '0040 PCT, each of which is incorporated herein by reference in its entirety. After the gas pressure within the gas chamber is reduced, a retraction spring (not shown) urges the carrier 3460 proximally.

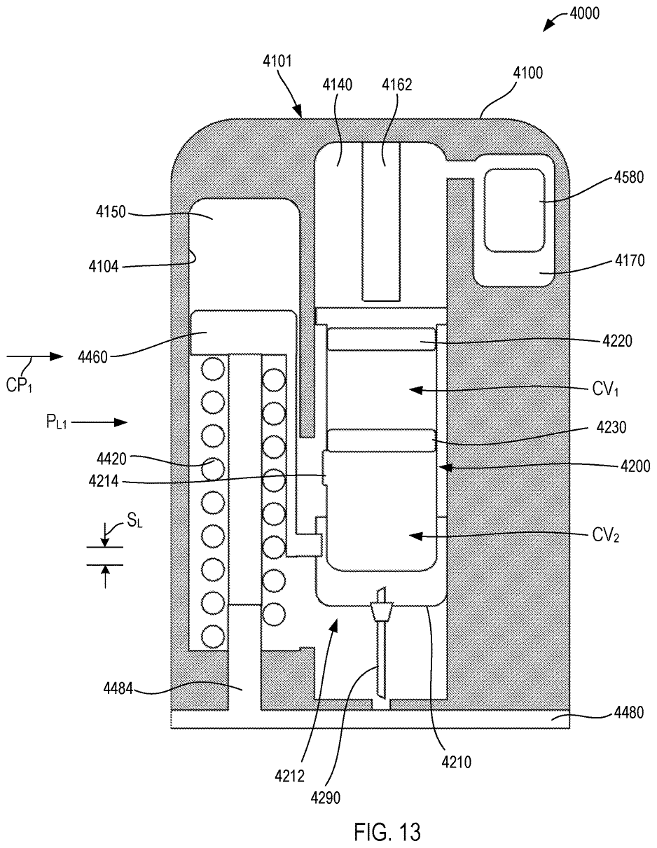

Although the medical injector 3000 is shown and described as using gas pressure to move the elastomeric member to cause mixing of the medicament, in some embodiments, the medical injector 1000 can have any other suitable mechanism for placing the device into a mixed configuration. For example, in some embodiment an autoinjector can have a mixing protrusion that is used to reconstitute a medicament and can also have a piston-less delivery system in which the force exerted by a pressurized gas can move the medicament container assembly relative to the housing and the elastomeric members relative to (e.g., within) the medicament container. For example, FIGS. 13-16 show a medical injector 4000 (also referred to as (medicament “delivery device,” “auto-injector,” “injector,” or “device”), according to an embodiment. The medical injector 4000 can be a gas-powered auto-injector configured to utilize a mixing protrusion in combination with a movement of the medicament container assembly to reconstitute the medicament contained within a medicament container assembly 4200 (e.g., prefilled syringe) and a compressed gas to deliver the medicament, as described herein. The medical injector 4000 can include any of the features, structures, and/or elements described above with reference to the medical injector 1000.

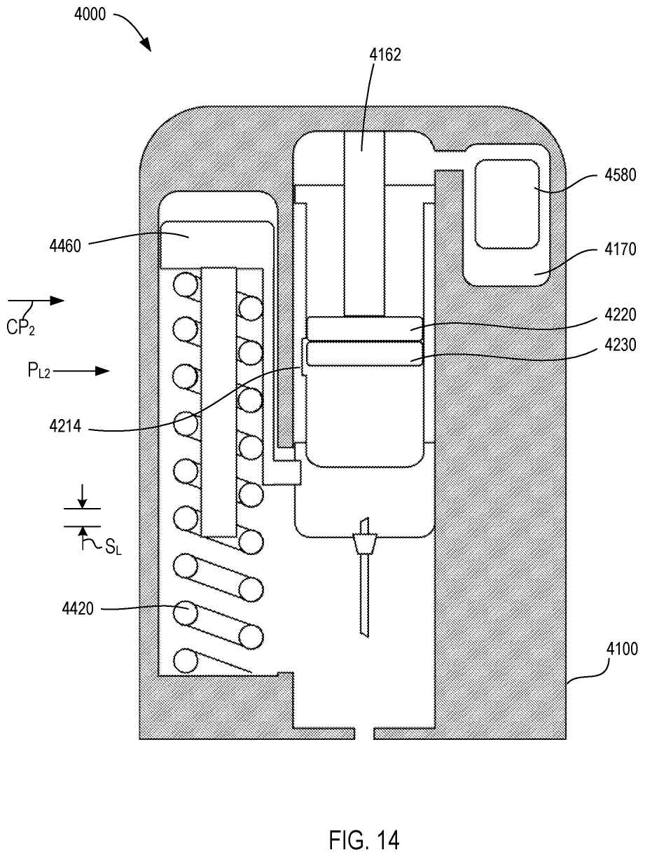

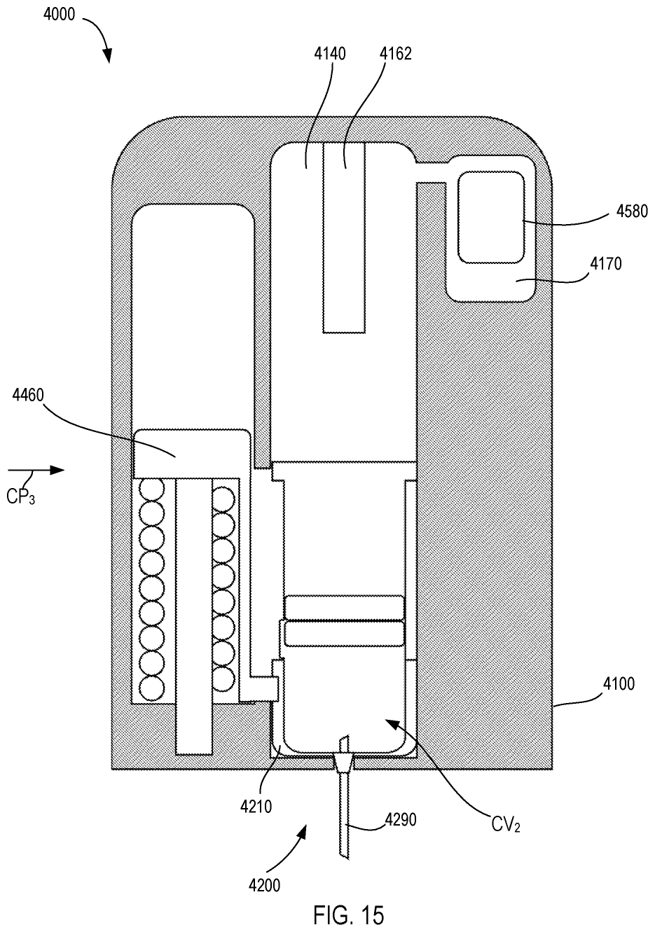

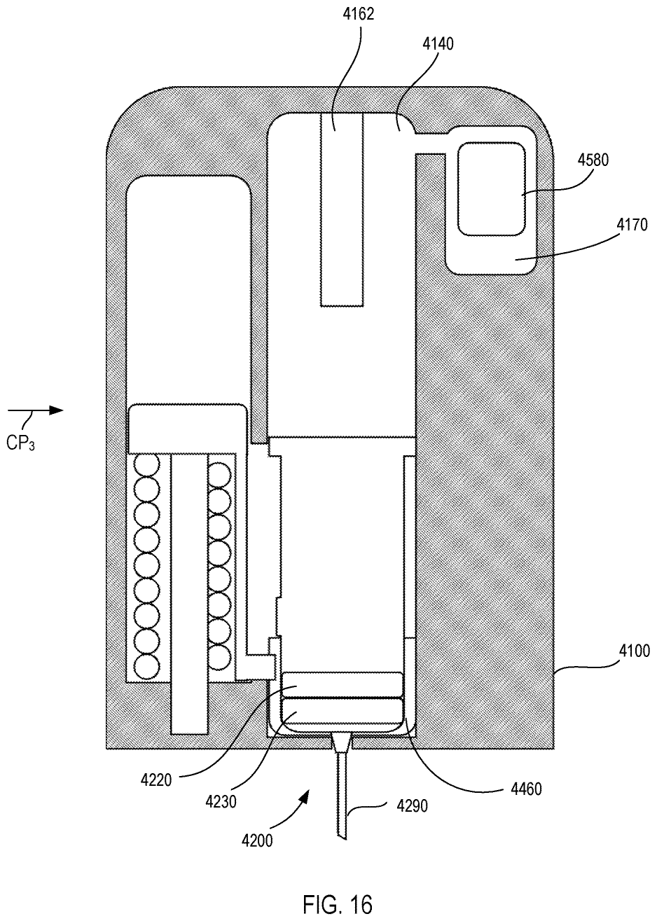

FIG. 13 depicts the medical injector 4000 in a first configuration. The first configuration can, for example, be a stored configuration. FIG. 14 depicts the medical injector 4000 following a transition from the first configuration to a second configuration. The second configuration can be a mixed configuration. The transition from the first configuration to the second configuration can be initiated by the removal of a safety member 4480 (FIG. 13). FIG. 15 depicts the medical injector 4000 following a transition from the second configuration to a third configuration. The third configuration can be an actuated configuration in which the medicament is reconstituted, a needle 4290 is in fluid communication with the reconstituted medicament within the container body 4210, and the needle 4290 is extended prior to dispensing the medicament from the medical injector 4000. FIG. 16 depicts the medical injector 4000 following a transition from the third configuration to a fourth configuration via the dispensing of the medicament from the medical injector 4000. In some embodiments, the fourth configuration can be followed by a transition to a fifth configuration in which the needle 4290 is retracted within the housing 4100.

As depicted, the medical injector 4000 includes a housing 4100 that has an inner wall 4104. The inner wall 4104 defines a medicament cavity 4140 and a mixing protrusion 4162. A medicament container assembly 4200 is disposed within the medicament cavity 4140. The medicament container assembly 4200 includes a container body 4210 that contains a dose of a medicament. The medicament container assembly 4200 has a first volume CV₁ and a second volume CV₂. The first volume CV₁ is fluidically isolated from the second volume CV₂ on a condition that the medical injector 4000 is in the first configuration, such as depicted in FIG. 13. The first volume CV₁ contains a first substance (e.g., a solvent), and the second volume CV₂ contains a second substance (e.g., a dry medicament component). A needle 4290 is axially aligned with the medicament container assembly 4200. The needle 4290 is positioned to have a separation S_{L} (e.g., a longitudinal separation) relative to a distal end portion 4212 of the container body 4210. The carrier 4460 at least partially surrounds the container body 4210 and the needle 4290. The carrier 4460 is movable (e.g., in the proximal direction) within the housing 4100 between a first carrier position CP₁ (as depicted in FIG. 13) and a second carrier position CP₂ (as depicted in FIG. 14). The carrier 4460 maintains the separation S_{L} between the needle 4290 and the distal end portion 4212 of the container body 4210 on a condition that the medical injector 4000 is in a stored condition (e.g., the first condition as depicted in FIG. 13) the carrier 4460 also maintains the separation S_{L} between the needle 4290 and the distal end portion 4212 of the container body 4210 on a condition that the medical injector 4000 is in a mixed condition (e.g., the second condition as depicted in FIG. 14). In other words, the carrier maintains the separation S_{L} of the needle 4290 a container body 4210 in both the first carrier position CP₁ and the second carrier position CP₂. The medicament container assembly 4200 (e.g., a first elastomeric member 4220) is in contact with the mixing protrusion 4162 on a condition that the carrier 4460 is at the second carrier position CP₂. Further, on the condition that the carrier 4460 is at the second carrier position CP₂, the first volume CV₁ is in fluid communication with the second volume CV₂.

The medicament container assembly 4200 can include any of the features, structures, and/or elements described herein with reference to the medicament container assembly 1200. The medicament container assembly 4200 is disposed within the medicament cavity 4140 and contains a dose of a medicament. The medicament container assembly includes the container body 4210, the first elastomeric member 4220 and a second elastomeric member 4230. The first elastomeric member 4220 is disposed within the proximal end portion of the container body 4210. The second elastomeric member 4230 is disposed within the container body 4210 distal to the first elastomeric member 4220. The first elastomeric member 4220 and the second elastomeric member 4230 define at least in part the first volume CV₁, while the second elastomeric member 4230 and a distal end portion of the container body 4210 defined the second volume CV₂. Additionally, the container body 4210 defines a bypass channel 4214. FIG. 13 depicts the second elastomeric member 4230 at a first longitudinal position PL₁ that results in the first volume CV₁ being fluidically isolated from the second volume CV₂. In contrast, FIG. 14 depicts the second elastomeric member 4230 following a distal movement (relative to the container body 4210) to a second longitudinal position PL₂ that is longitudinally aligned with the bypass channel 4214. On a condition that the second elastomeric member 4230 is at the second longitudinal position PL₂, the first volume CV₁ is in fluid communication with the second volume CV₂ via the bypass channel 4214.

As depicted in FIGS. 13-16, the mixing protrusion 4162 defined by the inner wall 4104 can be a rigid portion that extends into the medicament cavity 4140 from a proximal portion 4101 (e.g., a proximal end cap) of the housing 4100. The mixing protrusion 4162 can, as depicted, be axially aligned with the medicament container assembly 4200. The mixing protrusion 4162 is positioned to exert a longitudinal force on the first elastomeric member 4220 in response to the transition of the carrier 4460 from the first carrier position CP₁ to the second carrier position CP₂. The longitudinal force is configured to move the second elastomeric member 4230 from the first longitudinal position PL₁ (as depicted in FIG. 13) to the second longitudinal position PL₂ (as depicted in FIG. 14. Said another way, the proximal movement of the carrier 4460 from the first carrier position CP₁ toward the second carrier position CP₂ places the first elastomeric member 4220 into contact with the rigid mixing protrusion 4162. The resultant force exerted on the first elastomeric member 4220 by the mixing protrusion 4162 is greater than the friction force between the first elastomeric member 4220 and the container body 4210 and develops a distal movement of the first elastomeric member 4220 relative to the container body 4210. The distal movement of the first elastomeric member 4220 results in a corresponding distal movement of the second elastomeric member 4230, which, in turn, places the first volume CV₁ in fluid communication with the second volume CV₂ so that the medicament can be reconstituted. As depicted, the carrier 4460 maintains the separation S_{L} between the needle 4290 and the container body 4210 during the reconstitution of the medicament. In other words, the distal end portion 4212 of the container body 4210 is in a sealed configuration when the medical injector 4000 is in the mixed configuration.

As depicted in FIG. 13, in some embodiments, the medical injector 4000 includes a safety member 4480. The safety member 4480 is removably coupled to the housing 4100. The safety member 4480 includes a release member 4484. The release member 4484 is configured to actuate a spring 4420 coupled to the carrier 4460 on a condition that the safety member 4480 is decoupled from the housing 4100. Said another way, the removal of the safety member 4480 initiates a transition of the medical injector 4000 from the stored configuration to the mixed configuration by actuating the spring 4420 resulting in the reconstitution of the medicament within the medicament container assembly 4200. In other words, the medical injector 4000 is placed in a condition to be actuated to dispense the reconstituted medicament (e.g., a ready state) by the removal of the safety member 4480 and does not require additional inputs from an operator to prepare the medical injector 4000. The force exerted on the carrier 4460 by the spring 4420 (and thus the force exerted by the mixing protrusion 4162 on the first elastomeric member 4220) is less than a friction force between the carrier 4460 and the container body 4210. Accordingly, the container body 4210 remains in a fixed position relative to the carrier 4460 and the separation S_{L} of the needle 4290 from the container body 4210 is maintained in both the stored configuration and the mixed configuration.

As depicted in FIGS. 13-16, the medical injector 4000 includes an energy storage member 4580. The energy storage member for 4580 can be disposed within the housing 4100. For example, the energy storage member 4580 can be disposed in communication with a gas chamber 4170. The energy storage member 4580 is configured to produce a pressurized gas on a condition that the energy storage member 4580 is actuated to release a potential energy stored therein. For example, in some embodiments, the energy storage member can be a compressed gas cylinder having any desired pressure (and thus, mass) of gas therein. Upon actuation, the pressurized gas flows from the gas chamber 4170 to the medicament cavity 4140 to convey the medicament from the medicament container assembly 4200 (e.g., as depicted in FIG. 16). Said another way, the energy storage member 4580 is configured to produce a force to convey the medicament from the medicament container assembly 4200 on a transition of the medical injector 4000 from the mixed configuration to an actuated configuration, such as depicted in FIG. 15. In other words, the force of the pressurized gas can place the second volume CV₂ containing the reconstituted medicament in fluid communication with the needle 4290, extend the needle 4290 from the housing 4100, and move the first elastomeric member 4220 and the second elastomeric member 4230 in the distal direction to dispense the reconstituted medicament from the medicament container assembly 4200 as depicted in FIG. 16.

The container body 4210 is configured to move distally relative to the carrier 4460 in response to the force produced by the energy storage member 4580. The distal movement of the container body 4210 places the second volume CV₂ in fluid communication with the needle 4290 as depicted in FIG. 15. Said another way, the force produced by the energy storage member 4580 is greater than the friction force between the carrier 4460 and the container body 4210. As such, the container body 4210 can move in the distal direction relative to the carrier 4460 such that the separation S_{L} of the needle 4290 from the distal end portion 4212 of the container body 4210 is eliminated and the needle 4290 punctures the distal end portion 4212. Additionally, the carrier 4460 can move from the second carrier position CP₂ to a third carrier position CP₃ (as depicted in FIGS. 15 and 16) that is distal to both the first carrier position CP₁ and the second carrier position CP₂ in response to the force of the pressurized gas. On a condition that the carrier is in the third carrier position CP₃, the needle 4290 can extend from (e.g., outside) the housing 4100.

In response to the pressurized gas being conveyed into the medicament cavity 4140, the medicament container assembly 4200 is moved distally within the housing 4100 and/or relative to the needle 4290. Continued pressurization of the medicament cavity 4140 causes the needle 4290 and the medicament container assembly 4200 to move distally such that the needle 4290 extends from the housing (as shown in FIG. 15) to cause insertion of the needle at the target location. After extension of the needle 4290, continued pressurization of the medicament cavity 4140 causes the first elastomeric member 4220 and the second elastomeric member 4230 to move distally within the container body to convey the reconstituted medicament out of the second volume CV₂ via the needle 4290 (as shown in FIG. 16).

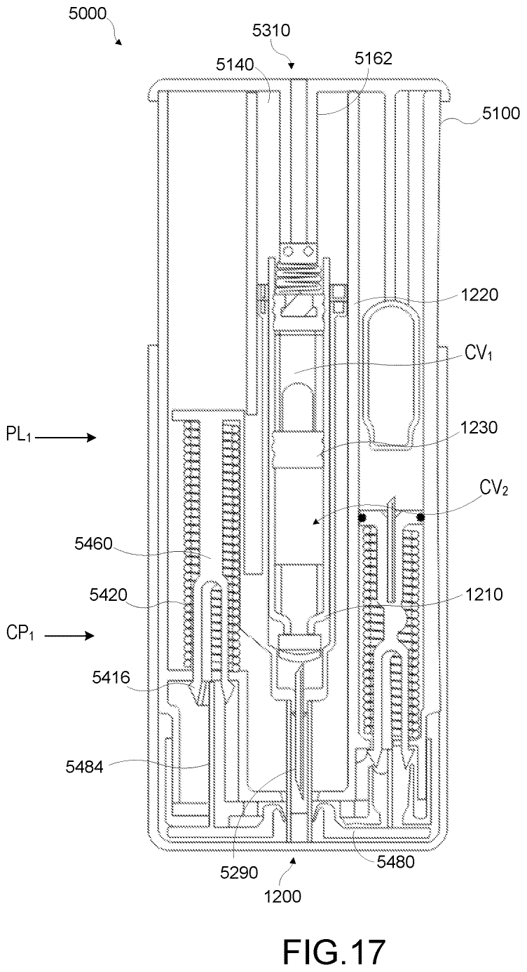

In some embodiments, the medicament container assembly 1200 described above can be a component of an autoinjector that has a mixing protrusion that is used to reconstitute a medicament. The autoinjector can also have a piston-less delivery system in which the force exerted by a pressurized gas can move the medicament container assembly relative to the housing and the elastomeric members relative to (e.g., within) the medicament container. For example, FIGS. 17-22 show a medical injector 5000 (also referred to as (medicament “delivery device,” “auto-injector,” “injector,” or “device”), according to an embodiment. The medical injector 5000 can be a gas-powered auto-injector configured to utilize a mixing protrusion in combination with a movement of the medicament container assembly to reconstitute the medicament contained within the medicament container assembly 1200. The medical injector 5000 can use a compressed gas to deliver the medicament, as described herein. The medical injector 5000 can include any of the features, structures, and/or elements described above with reference to any other medical injector described herein (e.g., the medical injector 1000 and the medical injector 4000).

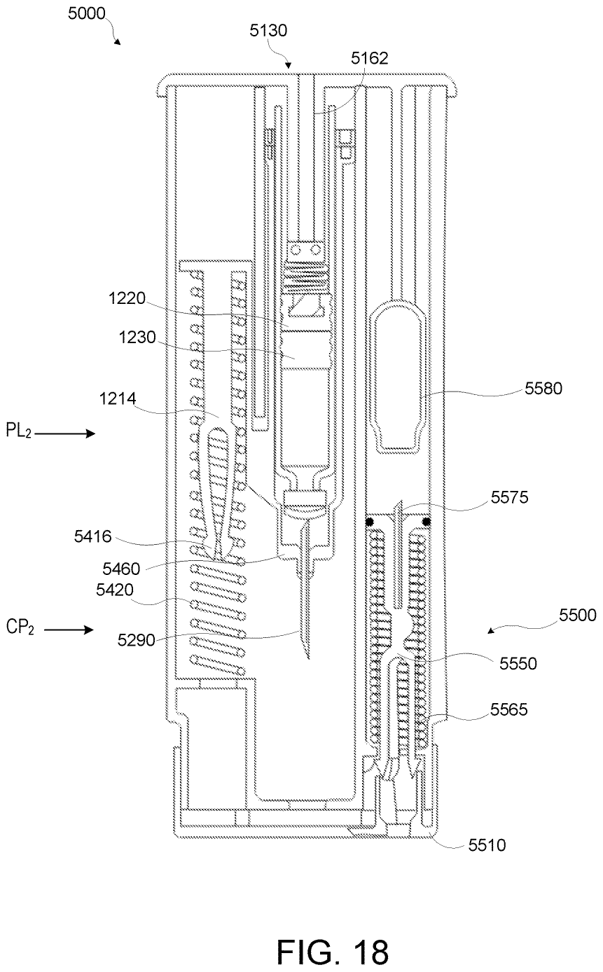

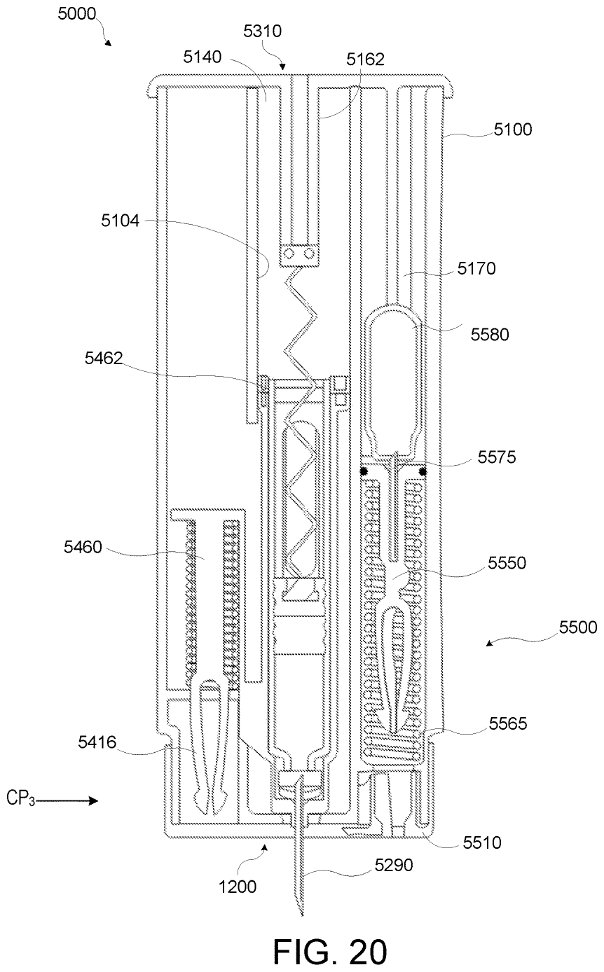

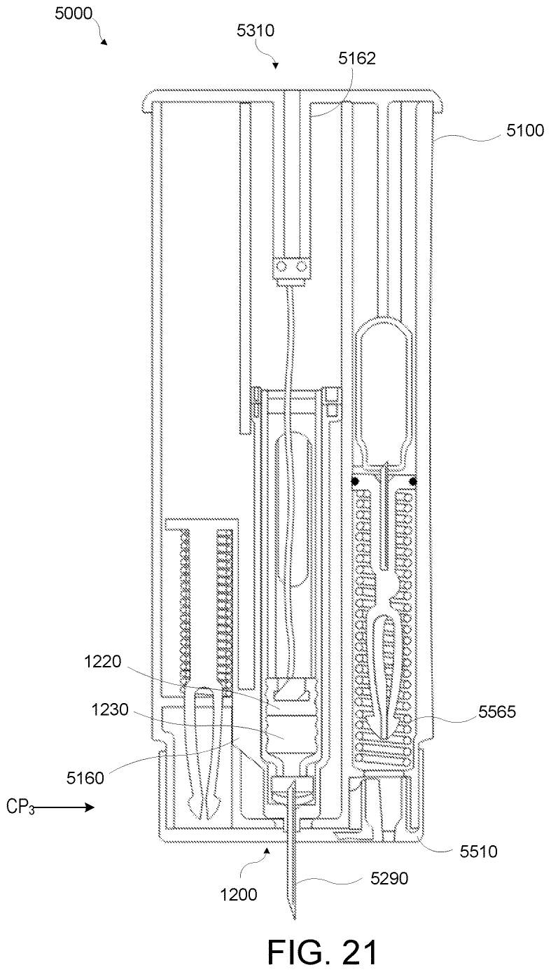

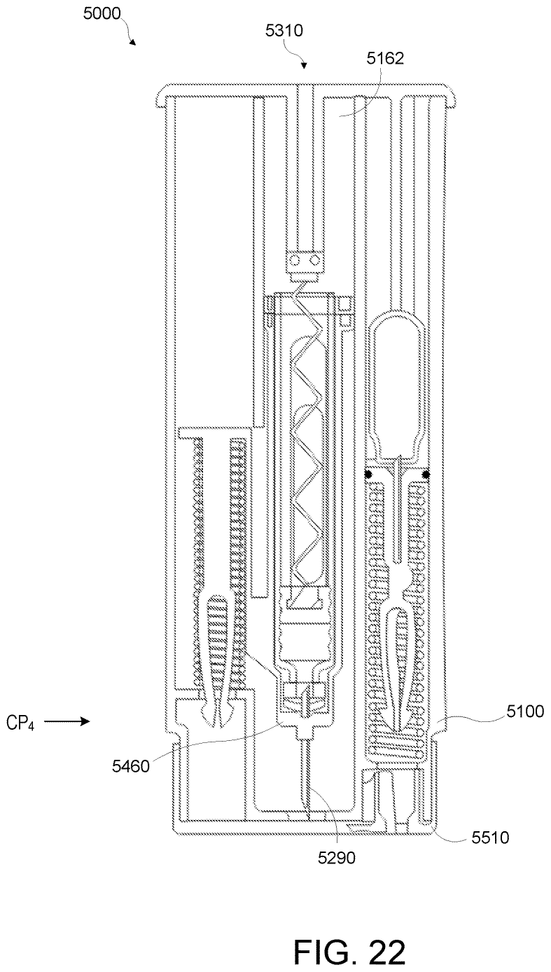

FIG. 17 depicts the medical injector 5000 in a first configuration. The first configuration can, for example, be a stored configuration. FIG. 18 depicts the medical injector 5000 following a transition from the first configuration to a second configuration. The second configuration can be a mixed configuration. The transition from the first configuration to the second configuration can be initiated by the removal of a safety member 5480. FIG. 20 depicts the medical injector 5000 following a transition from the second configuration to a third configuration. The third configuration can be an actuated configuration in which the medicament is reconstituted, a needle 5290 is in fluid communication with the reconstituted medicament within the container body 1210, and the needle 5290 is extended prior to dispensing the medicament from the medical injector 5000. FIG. 21 depicts the medical injector 5000 following a transition from the third configuration to a fourth configuration via the dispensing of the medicament from the medical injector 5000. FIG. 22 depicts the medical injector 5000 in a fifth (retracted) configuration in which the needle 5290 is retracted within the housing 5100.

As depicted, the medical injector 5000 includes a housing 5100 that has an inner wall 5104. The inner wall 5104 defines a medicament cavity 5140 and a mixing protrusion 5162. A medicament container assembly 5200 is disposed within the medicament cavity 5140. The medicament container assembly 5200 includes a container body 5210 that contains a dose of a medicament. The medicament container assembly 5200 has a first volume CV₁ and a second volume CV₂. The first volume CV₁ is fluidically isolated from the second volume CV₂ on a condition that the medical injector 5000 is in the first configuration, such as depicted in FIG. 13. The first volume CV₁ contains a first substance (e.g., a solvent), and the second volume CV₂ contains a second substance (e.g., a dry medicament component). A needle 5290 is axially aligned with the medicament container assembly 5200. The needle 5290 is positioned to have a separation S_{L} (e.g., a longitudinal separation) relative to a distal end portion 5212 of the container body 5210. A carrier 5460 at least partially surrounds the container body 5210 and the needle 5290. The carrier 5460 is movable (e.g., in the proximal direction) within the housing 5100 between a first carrier position CP₁ (as depicted in FIG. 17) and a second carrier position CP₂ (as depicted in FIG. 18). The carrier 5460 maintains the separation S_{L} between the needle 5290 and the distal end portion 5212 of the container body 5210 on a condition that the medical injector 5000 is in a stored condition (e.g., the first condition as depicted in FIG. 17) the carrier 5460 also maintains the separation S_{L} between the needle 5290 and the distal end portion 5212 of the container body 5210 on a condition that the medical injector 5000 is in a mixed condition (e.g., the second condition as depicted in FIG. 18). In other words, the carrier maintains the separation S_{L} of the needle 5290 a container body 5210 in both the first carrier position CP₁ and the second carrier position CP₂. The medicament container assembly 5200 (e.g., a first elastomeric member 5220) is in contact with the mixing protrusion 5162 on a condition that the carrier 5460 is at the second carrier position CP₂. Further, on the condition that the carrier 5460 is at the second carrier position CP₂, the first volume CV₁ is in fluid communication with the second volume CV₂.

The medicament container assembly 5200 can include any of the features, structures, and/or elements described herein with reference to the medicament container assembly 1200. The medicament container assembly 1200 is disposed within the medicament cavity 5140 and contains a dose of a medicament. The medicament container assembly includes the container body 5210, the first elastomeric member 5220 and a second elastomeric member 5230. The first elastomeric member 5220 is disposed within the proximal end portion of the container body 5210. The second elastomeric member 5230 is disposed within the container body 5210 distal to the first elastomeric member 5220. The first elastomeric member 5220 and the second elastomeric member 5230 define at least in part the first volume CV₁, while the second elastomeric member 5230 and a distal end portion of the container body 5210 defined the second volume CV₂. Additionally, the container body 5210 defines a bypass channel 5214. FIG. 17 depicts the second elastomeric member 5230 at a first longitudinal position PL₁ that results in the first volume CV₁ being fluidically isolated from the second volume CV₂. In contrast, FIG. 18 depicts the second elastomeric member 5230 following a distal movement (relative to the container body 5210) to a second longitudinal position PL₂ that is longitudinally aligned with the bypass channel 5214. On a condition that the second elastomeric member 5230 is at the second longitudinal position PL₂, the first volume CV₁ is in fluid communication with the second volume CV₂ via the bypass channel 5214.

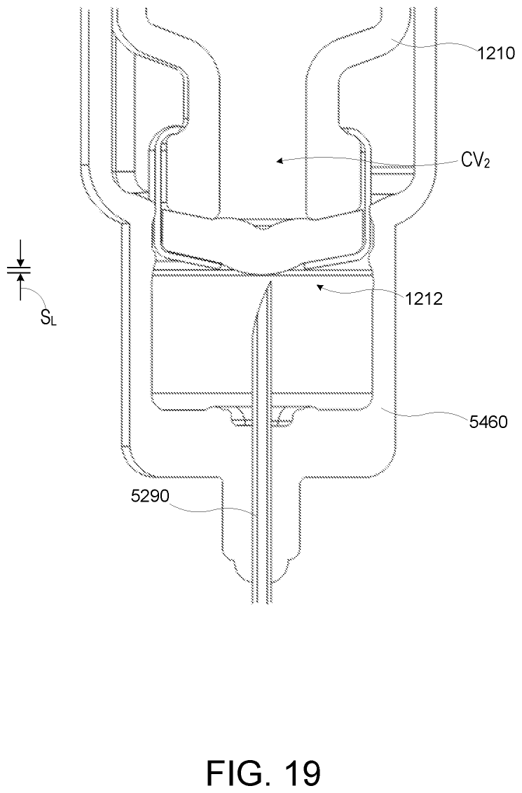

As depicted in FIGS. 17-22, the mixing protrusion 5162 defined by the inner wall 5104 can be a rigid portion that extends into the medicament cavity 5140 from a proximal portion 5101 (e.g., a proximal end cap) of the housing 5100. The mixing protrusion 5162 can, as depicted, be axially aligned with the medicament container assembly 5200. The mixing protrusion 5162 is positioned to exert a longitudinal force on the first elastomeric member 5220 in response to the transition of the carrier 5460 from the first carrier position CP₁ to the second carrier position CP₂. The longitudinal force is configured to move the second elastomeric member 5230 from the first longitudinal position PL₁ (as depicted in FIG. 13) to the second longitudinal position PL₂ (as depicted in FIG. 14. Said another way, the proximal movement of the carrier 5460 from the first carrier position CP₁ toward the second carrier position CP₂ places the first elastomeric member 5220 into contact with the rigid mixing protrusion 5162. The resultant force exerted on the first elastomeric member 5220 by the mixing protrusion 5162 is greater than the friction force between the first elastomeric member 5220 and the container body 5210 and develops a distal movement of the first elastomeric member 5220 relative to the container body 5210. The distal movement of the first elastomeric member 5220 results in a corresponding distal movement of the second elastomeric member 5230, which, in turn, places the first volume CV₁ in fluid communication with the second volume CV₂ so that the medicament can be reconstituted. As depicted, the carrier 5460 maintains the separation S_{L} (FIG. 19) between the needle 5290 and the container body 5210 during the reconstitution of the medicament. In other words, the distal end portion 5212 of the container body 5210 is in a sealed configuration when the medical injector 5000 is in the mixed configuration.

As depicted in FIG. 17, in some embodiments, the carrier 5460 includes a lock tab 5416. The lock tab 5416 can be a projection, a protrusion, and/or other structure having a surface configured to selectively engage the housing 5100. The lock tab 5416 is configured to retain the carrier 5460 at the first carrier position CP₁ on a condition that the medical injector 5000 is in the stored configuration. More specifically, the mixing actuator 5410 is disposed within the housing 5100 such that lock tab 5416 extends through an opening defined by the inner wall 5104 on a condition that the medical injector 5000 is in the stored configuration. Thus, the lock tab 5416 engages a portion of the inner wall 5104 and is maintained in contact therewith on the condition that the medical injector 5000 is in the stored configuration. In other words, lock tab 5416 engages a portion of the housing 5100 to limit proximal movement of the mixing actuator 5410 prior to actuation of a spring 5420 operably coupled to the carrier 5460.

In some embodiments, the medical injector 5000 includes a safety member 5480. The safety member 5480 is removably coupled to the housing 5100. The safety member 5480 includes a release member 5484 that is configured to actuate the spring 5420 on a condition that the safety member 5480 is decoupled from the housing 5100. As such, the engagement of the lock tab 5416 with the housing 5100 is maintained by the release member 5484 (which pushes the lock tabs 5416 away from each other and into contact with the housing 5100) on a condition that the safety member 5480 is coupled to the housing 5100. Said another way, the removal of the safety member 5480 initiates a transition of the medical injector 5000 from the stored configuration to the mixed configuration by actuating the spring 5420 resulting in the proximal movement of the carrier 5460 and the medicament container assembly 1200 and, thus, the reconstitution of the medicament within the medicament container assembly 1200. In other words, the medical injector 5000 is placed in a condition to be actuated to dispense the reconstituted medicament (e.g., a ready state) by the removal of the safety member 5480 and does not require additional inputs from an operator to prepare the medical injector 5000.

In some embodiments, the medical injector 5000 includes an energy storage member 5580. The energy storage member 5580 is disposed in communication with a gas chamber 5170. The energy storage member 5580 is configured to produce a pressurized gas on a condition that the energy storage member 5580 is actuated to release a potential energy stored therein. For example, in some embodiments, the energy storage member 5580 can be a compressed gas cylinder having any desired pressure (and thus, mass) of gas therein. Upon actuation, the pressurized gas flows from the gas chamber 5170 to the medicament cavity 5140 to move the carrier 5460 and the medicament container assembly 1200 in the distal direction and to convey the medicament from the medicament container assembly 1200.

As the gas flows into the medicament cavity 5140, the gas applies gas pressure to the upper portion of the carrier 5460 (including the seal member 5462 (e.g., a carrier seal)) and the first elastomeric member 1220 within the container body 1210. This gas force (the insertion force) causes the container body 1210, the carrier 5460 and the needle 5290 to contemporaneously move within the housing 5100 in the distal direction. The movement of the needle 5290 in a distal direction causes the distal end portion of the needle 5290 to exit the housing 5100 and enter the body of a patient prior to administering the medicament.

As depicted in FIGS. 17-22, the medicament container assembly 1200 is at least partially surrounded and supported by a carrier 5460. The carrier 5460 and the medicament container assembly 1200 are movable within the medicament cavity 5140 in the proximal direction and in the distal direction. For example, the carrier 5460 is at the first carrier position CP₁, as depicted in FIG. 17, when the medical injector 5000 is in the stored configuration. The carrier 5460 moves in the proximal direction to the second carrier position CP₂, as depicted in FIG. 18, on a transition of the medical injector from the stored condition to the mixed condition. The carrier 5460 distally from the second carrier position CP₂ to the third carrier position CP₃, as depicted in FIGS. 20 and 21 upon actuation of the medical injector 5000. The carrier 5460 can also move proximally from the third carrier position CP₃ to a fourth carrier position CP₄ (e.g., a retracted position) following the dispensing of the medicament from the medicament container assembly 1200.

In some embodiments, the carrier 5460 includes a seal member 5462 (FIG. 20) configured to form a substantially fluid tight seal with the inner wall 5104 of the housing 5100 defining the medicament cavity 5140. The seal member 5462 also seals the carrier 5460 and the container body 1210. Thus, the proximal portion of the medicament cavity 5140 forms a gas chamber that is substantially fluidically isolated by the seal member 5462. Specifically, the seal member 5462 has two sealing portions: one that forms a seal against the container body 1210 and the other that forms a seal against the inner surface of the housing 5100. In use, a pressurized gas applies an actuation force in a distal direction against the seal member 5462. The actuation force urges the two sealing portions outward (i.e., one sealing portion outward towards the container body 1210 and the other outward towards the housing 5100), which further enhances sealing. In other embodiments, however, the carrier 5460 can include multiple separate seals (e.g., O-rings) that form a fluid-tight seal with the housing 5100 and the container body 1210.

In some embodiments, the proximal portion of the medicament cavity 5140 can include a protrusion or stepped feature that limits movement of a retention portion of the carrier 5460. As depicted in FIG. 17-19, when the medical injector 5000 is in either the first configuration or the second configuration, the distal end portion of the container body 1210 is secured within a retention portion of the carrier 5460. Thus, the distal end portion 1212 of the medicament container assembly 1200 is within a coupling volume of the carrier 5460 but spaced apart from a proximal tip of the needle 5290, as depicted in FIG. 19. As shown in FIGS. 20 and 21, when the device is actuated the carrier 5460 moves distally, and the gas pressure exerted causes the medicament container assembly 1200 to be pushed distally and released from the retention portion (e.g., by an outward deformation of the retention portion).

In some embodiments, an optional proximal safety protrusion can contact the carrier in a manner to prevent an inadvertent release from the retention portion when the medical injector 5000 is in the first configuration and in the second configuration. Similarly stated, in some embodiments, the inner wall of the housing 5100 that defines the medicament cavity 5140 can include a stepped portion that engages the carrier 5460 near the retention portion. This arrangement limits the likelihood that retention portion will inadvertently be deformed outward when the medical injector 5000 is in the storage configuration and in the mixed configuration. This additional safety feature can be helpful to prevent the seal of the medicament container assembly 1200 from being punctured by the needle 5290 during conditions when the medical injector 5000 may be subjected to high external forces (e.g., vibratory forces, impact forces due to being dropped, etc.).

The interface between the carrier 5460 and the container body 1210 has a friction force within a specified range of magnitudes. The friction force has a magnitude that is greater than a force exerted on the container body 1210 via the carrier 5460 that results from the actuation of the spring 5420 operably coupled to the carrier 5460. Said another way, the friction force has a magnitude that is greater than any forces generated during the movement of the carrier 5460 from the first carrier position CP₁ to the second carrier position CP₂, including the longitudinal force exerted on the first elastomeric member 1220 by the mixing protrusion 5162. The friction force also has a magnitude that is less than the force exerted on the container body 1210 via the pressurized gas. Said another way, the force exerted on the container body 1210 by the pressurized gas from the energy storage member 5580 overcomes the friction force between the container body 1210 and the carrier 5460 resulting in a distal movement of the container body 1210 relative to the carrier 5460.

In some embodiments, a separation member (not shown) can be positioned between the distal end portion 1212 of the container body 1210 and the carrier 5460 in the coupling volume of the carrier 5460. The separation member can have a crush force that is within the specified range of magnitudes of the friction force. Accordingly, the separation member can preclude contact between the needle 5290 and the distal end portion 1212 of the container body 1210 her to the actuation of the energy storage member 5580.

As depicted in FIGS. 18 and 20, the medical injector 5000 includes a base 5510. The base 5510 has a similar structure and function as the bases shown in similar to that shown in the '4345 PCT and the '0040 PCT, each of which is incorporated herein by reference in its entirety. The medical injector 5000 can then be activated by moving the base 5510 from a first position, as depicted in FIG. 18, to a second position, as depicted in FIG. 20, to place the medical injector 5000 in the third (actuated) configuration. Similarly stated, the medical injector 5000 can be activated by the system actuator assembly 5500 (e.g., a puncturer release member 5550 and a spring 5565) by moving the base 5510 proximally relative to the housing 5100. The base 5510 is moved from its first position to its second position by placing the medical injector 5000 against a target surface (e.g., the body of the patient) and moving the base 5510 with respect to the housing 5100 in the proximal direction.

The proximal movement of the base 5510 from its first position to its second position actuates and/or otherwise releases the puncturer release member 5550. As such, the spring 5565 is allowed to transition from a first configuration (e.g., a compressed configuration) to a second configuration (e.g., a non-compressed configuration), thereby moving the puncturer release member 5550 within the gas chamber 5170. Proximal movement of the puncturer release member 5550 causes a puncturer 5575 to puncture and/or pierce a portion of the energy storage member 5580 (e.g., a frangible seal or the like). After the gas container 5580 has been punctured, an actuating portion of a compressed gas flows from the energy storage member 5580 (e.g., gas container), into the gas chamber 5170, and into the medicament cavity 5140. More specifically, with a seal of the puncturer release member 5550 forming a substantially fluid tight seal with the inner surface defining, at least in part, the gas chamber 5170, the actuating portion of the compressed gas fills the gas chamber 5170 and is forced through the gas passageway and into the medicament cavity 5140.

Insofar as the medical injector 5000 is configured to dispense the reconstituted medicament contained within the medicament container assembly 1200, the medical injector 5000 is actuated from the second configuration, such as depicted in FIG. 18. Accordingly, as depicted in FIG. 20, the first elastomeric member 1220 can be in contact with the second elastomeric member 1230 such that the gas pressure of the pressurized gas exerts a force sufficient to move both the elastomeric members 1220 and 1230 in the distal direction. The distal movement of the elastomeric members 1220 and 1230 generates a pressure upon the reconstituted medicament contained within the container body 1210, thereby allowing at least a portion of the medicament to flow out of the container body 1210 via the needle 5290, upon the transition from the third (actuated) configuration to the fourth (dispensed) configuration as shown in FIG. 21. Furthermore, when the elastomeric members 1220 and 1230 are disposed in a distal position within the container body 1210, the medical injector 5000 has delivered a dose of the reconstituted medicament.

As depicted in FIGS. 20 and 21, the medical injector 5000 includes a gas release assembly 5310. The gas release assembly 5310 expands as the first elastomeric member 1220 moves distally and serves to release a gas vent when the end of the injector stroke is reached. The gas release assembly 5310 a has similar structure and function as the gas release (or expandable) assemblies shown in the '4345 PCT and the '0040 PCT, each of which is incorporated herein by reference in its entirety.

FIG. 22 shows the medical injector 5000 in its retracted position (e.g., the fifth configuration). The retraction of the carrier 5460 and the needle 5290 occurs in response to the pressurized gas within the medicament cavity 5140 of the housing 5100 being vented by the expansion of the gas release assembly 5310. Specifically, when the first elastomeric member 1220 reaches the desired position (i.e., its distal-most position corresponding to the end of injection), the gas release assembly 5310 actuates a valve to release the gas. Operation of the gas release assembly 5310 can be similar to that shown in the '4345 PCT and the '0040 PCT, each of which is incorporated herein by reference in its entirety. After the gas pressure within the gas chamber is reduced, a retraction spring (not shown) urges the carrier 5460 proximally.

While various embodiments have been described above, it should be understood that they have been presented by way of example only, and not limitation. Where schematics and/or embodiments described above indicate certain components arranged in certain orientations or positions, the arrangement of components may be modified. While the embodiments have been particularly shown and described, it will be understood that various changes in form and details may be made. Although various embodiments have been described as having particular features and/or combinations of components, other embodiments are possible having a combination of any features and/or components from any of embodiments as discussed above.

Where methods and/or events described above indicate certain events and/or procedures occurring in certain order, the ordering of certain events and/or procedures may be modified. Additionally, certain events and/or procedures may be performed concurrently in a parallel process when possible, as well as performed sequentially as described above.

Although the medicament delivery devices are shown and described herein as being medical injectors having a medicament container divided into two portions, in other embodiments, any of the components, methods and/or formulations described herein can be used in any suitable medicament delivery device. In some embodiments, the medicament delivery device can include a medicament container having any number of plungers and/or defining any number of volumes therein.

Although the components and methods described herein are shown and described as being included in devices that include a medicament, in other embodiments, any of the components and/or methods described herein can be used in either an actual medicament delivery device or a simulated medicament delivery device. A simulated medicament delivery device, for example, can correspond to an actual medicament delivery device and can facilitate the training of a user in the operation of the corresponding actual medicament delivery device. A simulated medicament delivery device or trainer can be similar to the simulated medicament delivery devices or trainers described in U.S. Patent Publication Number 2008/0059133, entitled “Medical Injector Simulation Device,” filed Feb. 27, 2007, which is incorporated herein by reference in its entirety.

In such embodiments, the simulated medicament delivery device can simulate the actual medicament delivery device in any number of ways. For example, in some embodiments, the simulated medicament delivery device can have a shape corresponding to a shape of the actual medicament delivery device, a size corresponding to a size of the actual medicament delivery device and/or a weight corresponding to a weight of the actual medicament delivery device. Moreover, in some embodiments, the simulated medicament delivery device can include components that correspond to the components of the actual medicament delivery device. In this manner, the simulated medicament delivery device can simulate the look, feel, forces, and sounds of the actual medicament delivery device. For example, in some embodiments, the simulated medicament delivery device can include external components that correspond to external components of the actual medicament delivery device. In some embodiments, the simulated medicament delivery device can include internal components that correspond to internal components of the actual medicament delivery device.

In some embodiments, however, the simulated medicament delivery device can be devoid of a medicament and/or those components that cause the medicament to be delivered (e.g., a needle, a nozzle or the like). In this manner, the simulated medicament delivery device can be used to train a user in the use of the actual medicament delivery device without exposing the user to a needle and/or a medicament. Moreover, the simulated medicament delivery device can have features to identify it as a training device to prevent a user from mistakenly believing that the simulated medicament delivery device can be used to deliver a medicament. For example, in some embodiments, the simulated medicament delivery device can be of a different color than a corresponding actual medicament delivery device. Similarly, in some embodiments, the simulated medicament delivery device can include a label and/or other indicia clearly identifying it as a training device.

Any of the medicament containers described herein can be any container suitable for storing the compositions disclosed herein. In some embodiments, the medicament container can be a pre-filled syringe, a pre-filled cartridge, a vial, or the like. In some embodiments, for example, any of the devices shown and described herein can include components and/or mechanisms to accommodate a pre-filled syringe, similar to the embodiments shown and described in U.S. Patent Publication No. 2013/0023825 entitled, “Medicament Delivery Devices for Administration of Medicament within a Prefilled Syringe,” filed Jan. 25, 2012 the disclosure of which is incorporated herein by reference in its entirety. In other embodiments, the medicament containers described here can be a container having a flexible wall, such as, for example, a bladder.

Any of the devices and/or medicament containers shown and described herein can be included in a kit (not shown), which can include fungible components and reusable components. For example, in some embodiments, at least a housing of a medical injector can be reusable without the need for sterilization, as described in detail above. In some embodiments, such as with the medical injector 1000, the proximal cap 1160 can be removed from the housing 1100 to allow access to and removal of the used components disposed within the housing 1100. In addition, the removal of the proximal cap 1160 from the housing 1100 can allow for any suitable portion of the medical injector 1000 to be reset to, for example, a pre-activated or pre-actuated configuration, as described above.

### Medicaments and Formulations

Any of the devices and/or medicament containers shown and described herein can include any suitable medicament or therapeutic agent. For example, although the medical injectors described above are shown and described as including a multi-chamber medicament container (e.g., container body 1210) that includes a substantially dry medicament and a solvent in other embodiments, any of the medicament delivery devices disclosed herein can include a multi-chamber container that is filled with any suitable substances. For example, in some embodiments, any of the medicament delivery devices disclosed herein can include a medicament container (e.g., a cartridge) that separately stores and mixes, upon actuation, two liquid substances. For example in some embodiments, any of the devices shown and described herein can include a medicament container filled with (in separate chambers) epinephrine and at least one antihistamine (e.g., epinephrine and diphenhydramine, epinephrine and hydroxyzine, epinephrine and cetirizine); an antipsychotic medicament and a benzodiazepine (e.g. haloperidol and diazepam, haloperidol and midazolam, haloperidol and lorazepam); insulin and a GLP-1 analog or incretin mimetic (e.g. insulin and exenatide, insulin and lixisenatide); an NSAID and an opiode (e.g., ketorolac and buprenorphine). Other suitable compositions that can be included in any of the medicament containers and/or devices described herein include pralidoxime chloride and atropine; obidoxime chloride and atropine; epinephrine and atropine; methotrexate and etanercept; methotrexate and adalimumab; and methotrexate and certolizumab.

In some embodiments, a dry medicament composition can include a dose of atropine base of between about 1.5 mg and about 1.7 mg of atropine base. In other embodiments, a dry medicament composition can include a dose of atropine base of between about 1.9 mg and about 2.1 mg of atropine sulfate. In such embodiments, the drug product can include a sufficient amount of solvent to produce a single dose of about 2 mL of drug solution.

In some embodiments, a composition can include glucagon and/or any pharmaceutically acceptable constituents for use in the medicament delivery devices disclosed herein. In some embodiments, the glucagon formulation can be prepared and/or filled according to any suitable method such as, for example, those described in U.S. Patent Publication No. 2013/0023822 incorporated by reference hereinabove. A composition according to an embodiment can be formulated such that the target concentration of glucagon in the solution, either before lyophilization and/or after being reconstituted upon actuation of the device, is approximately 1 mg/mL. In other embodiments, the target concentration of glucagon in the solution, either before lyophilization and/or after being reconstituted, can be approximately 2 mg/mL, approximately 1.5 mg/mL, approximately 0.5 mg/ml (e.g., a pediatric dose) or approximately 0.25 mg/mL. In other embodiments, a composition can be formulated such that the target concentration of glucagon in the solution, either before lyophilization and/or after being reconstituted upon actuation of the device, is between approximately 0.25 mg/mL and 2 mg/mL, between approximately 0.5 mg/mL and 1 mg/mL, or between approximately 0.8 mg/mL and 1.2 mg/mL.

In certain embodiments, the concentration (either before lyophilization or upon reconstitution) of glucagon in a glucagon formulation is about 1 mg/mL and the total solute concentration is about 50 mg/mL. For example, in some embodiments, a composition can include glucagon and any suitable bulking agents to increase the total solute concentration in the glucagon formulation. In this manner, the glucagon formulation can be more effectively lyophilized and/or reconstituted. For example, in some embodiments, as described below, certain bulking agents can be used to improve the stability, solubility and/or efficacy of the composition when reconstituted in any of the devices shown and described herein. In some embodiments, certain bulking agents can be used to produce a visual indicia when the composition is reconstituted (e.g., such agents can allow the reconstituted medicament to be more easily detected by the user).

In some embodiments, a composition can include a peptide, such as, for example, glucagon and a carbohydrate. In this manner, the stability of the peptide (e.g., glucagon) can be increased during lyophilization and subsequent storage. In particular, the stability of peptides, such as glucagon, can be increased in an amorphous (i.e. non-crystalline) environment. It is believed that carbohydrates undergoing dehydration create a solid-state environment that is amorphous and exhibits high viscosity when maintained below the glass transition temperature. In addition, carbohydrates contain multiple hydroxyl groups that may form hydrogen bonds with polar groups on a protein or peptide surface in an amorphous solid-state environment. Without being bound by any particular mechanism, when water is removed during lyophilization, such carbohydrates may maintain the hydrogen bonds and preserve the native-like solid state of the polypeptide structure. In certain embodiments, therefore, the glucagon formulations include other excipients, such as, but not limited to carbohydrates. Suitable carbohydrates include, but are not limited to, lactose, trehalose, mannitol, and combinations thereof.

Additionally, the solubility of glucagon increases below a pH of 4. In certain embodiments, the glucagon formulations, prior to lyophilization and/or after reconstitution, have a pH of less than about pH 5.0, including less than about pH 4.5, less than about pH 4.0, less than about pH 3.5, less than about pH 3.0, less than about pH 2.5, less than about pH 2.0. In other embodiments of the invention, the glucagon formulations, prior to lyophilization and/or after reconstitution, have a pH range of about pH 1.5 to about pH 5.0, inclusive of all ranges and subranges therebetween, e.g., about pH 2.0 to about pH 4.5, about pH 2.0 to about pH 4.0, about pH 2.0 to about pH 3.5, about pH 2.0 to about pH 3.0, about pH 2.0 to about pH 2.5, about pH 2.5 to about pH 4.5, about pH 2.5 to about pH 4.0, about pH 2.5 to about pH 3.5, about pH 2.5 to about pH 3.0, about pH 3.0 to about pH 4.5, about pH 3.0 to about pH 4.0, about pH 3.0 to about pH 3.5, about pH 3.5 to about pH 4.5, and about pH 3.5 to about pH 4.0. In certain embodiments, the pH of the glucagon formulation is adjusted prior to lyophilization by the addition of a suitable acid, such as hydrochloric acid or citric acid.

The lyophilized or dry formulations of the present invention may be reconstituted by any suitable solvent or combination of solvent, including, but not limited to, water, sterile water, glycerin, or hydrochloric acid.

As described above, in some embodiments, a dry formulation can include any suitable bulking agents and/or excipients.

In some embodiments, the medicament contained within any of the medicament containers shown herein can be a vaccine, such as, for example, an influenza A vaccine, an influenza B vaccine, an influenza A (H1N1) vaccine, a hepatitis A vaccine, a hepatitis B vaccine, a haemophilus influenza Type B (HiB) vaccine, a measles vaccine, a mumps vaccine, a rubella vaccine, a polio vaccine, a human papilloma virus (HPV) vaccine, a tetanus vaccine, a diphtheria vaccine, a pertussis vaccine, a bubonic plague vaccine, a yellow fever vaccine, a cholera vaccine, a malaria vaccine, a smallpox vaccine, a pneumococcal vaccine, a rotavirus vaccine, a varicella vaccine and/or a meningococcus vaccine. In other embodiments, the medicament contained within any of the medicament containers shown herein can be epinephrine. In other embodiments, the medicament contained within any of the medicament containers shown herein can be naloxone, including any of the naloxone formulations described in U.S. patent application Ser. No. 13/036,720, entitled “Medicament Delivery Device for Administration of Opioid Antagonists Including Formulation for Naloxone,” filed on Feb. 28, 2011, the disclosure of which is incorporated herein by reference in its entirety.

In other embodiments, the medicament contained within any of the medicament containers shown herein can include insulin, glucagon, human growth hormone (HGH), erythropoiesis-stimulating agents (ESA), DeMab, Interferon and other chronic therapies, or the like. Such formulations can be produced using a general lyophilization process with glucagon (of recombinant origin) using bulking agents, stabilizers, buffers, acidifying agents or other excipients comprising of, but not limited to, one or more of the following combinations: lactose, hydrochloric acid; glucose, histidine, hydrochloric acid; trehalose, mannitol, citrate; trehalose, mannitol, hydrochloric acid; trehalose, glycine, hydrochloric acid; Mannitol, ascorbic acid; and Glycine, hydrochloric acid.

In other embodiments any of the injectors described herein can be filled with and/or used to inject medicament formulations, including lyophilized biologics and/or biopharmaceuticals, such as, for example, canakinumab, certolizumab, golimumab, and/or interleukins, for the treatment of crypyrin associated periodic syndromes, hereditary andioedema, and other auto-immune diseases. In yet other embodiments any of the injectors described herein can be filled with and/or used to inject intranasal biologics, such as glucagon or human growth hormone, formulated for use in an auto injector, for the treatment of musculoskeletal diseases, growth disorders, diabetes & treatment related disorders.

In other embodiments, any of the injectors described herein can be filled with and/or used to inject an anti-thrombotics, such as LMWH, ULMWH, Xa Inhibitors, biotinylated idraparinux, etc., for either the acute management and/or surgical prophylaxis of deep vein thrombosis and/or pulmonary embolism or for the management of other conditions which may require anticoagulation to prevent thromboembolism, such as its use in cardiovascular diseases including atrial fibrillation and ischemic stroke. In another example, in some embodiments an injector according to an embodiment can be filled with and/or used to inject formulations for the treatment of asthma and/or chronic obstructive pulmonary disease.

In other embodiments, any of the injectors described herein can be filled with and/or used to inject recombinant hyaluronidase.

In other embodiments, any of the injectors described herein can be filled with and/or used to inject depot medroxyprogesterone acetate for the treatment of infertility.

In other embodiments, any of the injectors described herein can be filled with and/or used to inject environmental, food, and household allergen formulations for the treatment of allergic disease, specifically for use in immunotherapy.

In still other embodiments, the medicament contained within any of the medicament containers shown herein can be a placebo substance (i.e., a substance with no active ingredients), such as water.

The medicament containers and/or medicament delivery devices disclosed herein can contain any suitable amount of any medicament. For example, in some embodiments, a medicament delivery device as shown herein can be a single-dose device containing an amount of medicament to be delivered of approximately 0.4 mg, 0.8 mg, 1 mg, 1.6 mg or 2 mg.

What is claimed is:

1. An apparatus comprising:
a housing having an inner wall that defines a first gas chamber, a second gas chamber, and a medicament cavity, the medicament cavity being in fluid communication with the first gas chamber and the second gas chamber;
a medicament container assembly disposed within the medicament cavity, the medicament container assembly containing a medicament, the medicament container assembly having a first volume and a second volume, the first volume being fluidically isolated from the second volume, the first volume containing a first substance, the second volume containing a second substance;
a mixing actuator positioned within the first gas chamber, the mixing actuator being movable within the first gas chamber between a first mixing actuator position and a second mixing actuator position, movement of the mixing actuator from the first mixing actuator position towards the second mixing actuator position causing production of a first pressurized gas to place the first volume in fluid communication with the second volume; and
an energy storage member disposed in communication with the second gas chamber, the energy storage member being configured to produce a second pressurized gas to convey the medicament from the medicament container assembly on a condition that the energy storage member is actuated to release a potential energy stored therein.

2. The apparatus of claim 1, wherein:
the first gas chamber has a first pneumatic volume on a condition that the mixing actuator is at the first mixing actuator position;
the first gas chamber has a second pneumatic volume on a condition that the mixing actuator is at the second mixing actuator position; and
the second pneumatic volume is less than the first pneumatic volume.

3. The apparatus of claim 1, wherein:
an air within the first gas chamber has a first pressure on a condition that the mixing actuator is at the first mixing actuator position;
the air within the first gas chamber has a second pressure on a condition that the mixing actuator is at the second mixing actuator position; and
the second pressure is greater than the first pressure.

4. The apparatus of claim 1, further comprising:
a spring coupled between the housing and a distal end portion of the mixing actuator, the spring configured to urge the mixing actuator towards the second mixing actuator position.

5. The apparatus of claim 4, wherein:
the mixing actuator includes a lock tab; and
the lock tab is positioned to engage the housing to retain the mixing actuator at the first mixing actuator position.

6. The apparatus of claim 5, further comprising:
a safety member removably coupled to the housing, the safety member including a release member configured to actuate the spring on a condition that the safety member is decoupled from the housing.

7. The apparatus of claim 1, further comprising:
an actuator seal surrounding a proximal end portion of the mixing actuator, the actuator seal being positioned between the proximal end portion of the mixing actuator and a wall of the first gas chamber.

8. The apparatus of claim 1, further comprising:
a one-way valve positioned fluidically between the first gas chamber and the medicament cavity, the one-way valve being configured to preclude a flow of gas from the medicament cavity into the first gas chamber.

9. The apparatus of claim 1, wherein:
the medicament container assembly includes a container body, a first elastomeric member, and a second elastomeric member;
the first elastomeric member is disposed within a proximal end portion of the container body;
the second elastomeric member is disposed within the container body distal to the first elastomeric member;
the first elastomeric member and the second elastomeric member define at least in part the first volume containing the first substance; and
the second elastomeric member and a distal portion end portion of the container body define the second volume containing the second substance.

10. Apparatus of claim 9, wherein:
the container body defines a bypass channel;
the first volume is fluidically isolated from the second volume on a condition that the second elastomeric member is at a first longitudinal position;
the first volume is fluidically coupled to the second volume on a condition that the second elastomeric member is at a second longitudinal position;
the second longitudinal position is distal to the first longitudinal position; and
the second longitudinal position is longitudinally aligned with the bypass channel.

11. An apparatus comprising:
a housing having an inner wall that defines a medicament cavity and a mixing protrusion;
a medicament container assembly disposed within the medicament cavity, the medicament container assembly including a container body containing a medicament, the medicament container assembly having a first volume and a second volume, the first volume being fluidically isolated from the second volume, the first volume containing a first substance, the second volume containing a second substance;
a needle axially aligned with the medicament container assembly, the needle having a separation from a distal end portion of the container body; and
a carrier at least partially surrounding the container body and the needle, the carrier being movable within the housing between a first carrier position and a second carrier position, the carrier maintaining the separation on a condition that the apparatus is in a stored condition and on a condition that the apparatus is in a mixed condition, wherein the medicament container assembly is in contact with the mixing protrusion and the first volume is in fluid communication with the second volume on a condition that the carrier is at the second carrier position.

12. The apparatus of claim 11, wherein the mixing protrusion is axially aligned with the medicament container assembly.

13. The apparatus of claim 11, wherein:
the medicament container assembly further includes a first elastomeric member and a second elastomeric member;
the first elastomeric member is disposed within a proximal end portion of the container body;
the second elastomeric member is disposed within the container body distal to the first elastomeric member:
the first elastomeric member and the second elastomeric member define at least in part the first volume containing the first substance; and
the second elastomeric member and a distal end portion of the container body define the second volume containing the second substance.

14. Apparatus of claim 13, wherein:
the container body defines a bypass channel;
the first volume is fluidically isolated from the second volume on a condition that the second elastomeric member is at a first longitudinal position;
the first volume is fluidically coupled to the second volume on a condition that the second elastomeric member is at a second longitudinal position;
the second longitudinal position is distal to the first longitudinal position; and
the second longitudinal position is longitudinally aligned with the bypass channel.

15. The apparatus of claim 14, wherein:
the mixing protrusion is positioned to exert a longitudinal force on the first elastomeric member in response to a transition of the carrier from the first carrier position to the second carrier position; and
the longitudinal force is configured to move the second elastomeric member from the first longitudinal position to the second longitudinal position.

16. The apparatus of claim 11, further comprising:
an energy storage member disposed within the housing, the energy storage member being configured to produce a force to convey the medicament from the medicament container assembly on a transition of the apparatus from the mixed condition to an actuated condition.

17. The apparatus of claim 16, wherein:
the container body is configured to develop a distal movement relative to the carrier in response to the force produced by the energy storage member; and
the distal movement of the container body places the second volume in fluid communication with the needle.

18. The apparatus of claim 11, wherein:
the carrier includes a lock tab; and
the lock tab is positioned to engage the housing to retain the carrier at the first carrier position.

19. The apparatus of claim 18, further comprising:
a safety member removably coupled to the housing, the safety member including a release member configured to actuate a spring operably coupled to the carrier on a condition that the safety member is decoupled from the housing, the actuation of the spring transitioning the apparatus from the stored condition to the mixed condition.
